(19) United States
(12) Patent Application Publication
(10) Pub. No.: US 2019/0167579 A1
(43) Pub. Date: Jun. 6, 2019

(54) ITRACONAZOLE DRY POWDERS

(71) Applicant: Pulmatrix Operating Company, Inc., Lexington, MA (US)

(72) Inventors: Jason M. Perry, Cambridge, MA (US); Jean C. Sung, Cambridge, MA (US); David L. Hava, Natick, MA (US)

(21) Appl. No.: 16/173,744

(22) Filed: Oct. 29, 2018

Related U.S. Application Data
(60) Provisional application No. 62/578,148, filed on Oct. 27, 2017.

(51) Int. Cl. A61K 9/00; A61K 31/496; A61K 47/02; A61K 47/18; A61K 47/26; A61P 31/10; A61P 11/00

(52) U.S. Cl. CPC A61K 9/0075; A61K 31/496; A61K 47/02; A61P 11/00; A61K 47/26; A61P 31/10; A61K 47/183

(57) ABSTRACT

In one aspect, a dry powder comprising respirable dry particles that comprise amorphous itraconazole in an amount of about 45% to about 75%, sodium sulfate in an amount of about 10% to about 55%, and optionally one or more other excipients, wherein all percentages are weight percentages on a dry basis and all the components of the respirable dry particles amount to 100%. In another aspect, a dry powder comprising respirable dry particles that comprise amorphous itraconazole in an amount of about 45% to about 55%, sodium chloride in an amount of about 30% to about 40%, and leucine in an amount of about 10% to about 20%, wherein all percentages are weight percentages on a dry basis and all the components of the respirable dry particles amount to 100%.

### RELATED APPLICATIONS

This application claims the benefit of U.S. Application No. 62/578,148, filed Oct. 27, 2017. The entire teachings of the above application are incorporated herein by reference.

### BACKGROUND

Pulmonary Aspergillus ssp. infections are a growing concern in asthma and cystic fibrosis (CF) patients, both in those with chronic infection and in patients with Allergic Bronchopulmonary Aspergillosis (ABPA), a severe inflammatory condition that requires long course of oral steroids. Triazoles, including itraconazole, are commonly administered as oral or intravenous (IV) formulations as treatments for both pulmonary infection and ABPA. Existing oral formulations of itraconazole are limited by poor oral bioavailability, adverse side effects, and extensive drug-drug interactions. Pulmonary delivery of itraconazole offers an opportunity to overcome these limitations and achieve high lung concentrations of drug, while minimizing systemic exposure and thus side effects.

The chemical structure of itraconazole was first described in U.S. Pat. No. 4,916,134. Itraconazole is a triazole anti-fungal agent providing therapeutic benefits, e.g., in the treatment of fungal infections, and is the active ingredient in SPORANOX (itraconazole) which may be delivered orally or intravenously. Itraconazole can be synthesized using a variety of methods which are well known in the art.

### SUMMARY OF THE INVENTION

In one aspect, a dry powder containing respirable dry particles that contains itraconazole in an amount of about 45% to about 75%, sodium sulfate in an amount of about 10% to about 55%, and optionally one or more other excipients, where all percentages are weight percentages on a dry basis and all the components of the respirable dry particles amount to 100%. The option excipient may be leucine, or it may be mannitol, or it may be a combination of the two. The itraconazole may be about 45% to about 55%, or about 50%. The sodium sulfate may be about 30% to about 40%, or about 35%. The other excipients, when present, may be about 5% to about 25%, or about 15%. In one embodiment, the dry powder is a respirable dry powder.

In another aspect, a dry powder contains respirable dry particles that contain itraconazole in an amount of about 45% to about 55%, sodium chloride in an amount of about 30% to about 40%, and leucine in an amount of about 10% to about 20%, where all percentages are weight percentages on a dry basis and all the components of the respirable dry particles amount to 100%. The itraconazole is preferably in an amount of about 50%. The sodium chloride is preferably in an amount of about 35%. The leucine is preferably in an amount of about 15%. In one embodiment, the dry powder is a respirable dry powder.

In either aspect, the respirable dry particles may have a volume median geometric diameter (VMGD) about 10 microns or less, or preferably, about 5 microns or less; a tap density of about 0.2 g/cc or greater, between 0.2 g/cc and 1.0 g/cc, or greater that 0.4 g/cc; an MMAD of between about 1 micron and about 5 microns; a 1/4 bar dispersibility ratio (1/4 bar) or 0.5/4 bar dispersibility ratio (0.5/4 bar) of less than about 1.5, preferably less than about 1.3, as measured by laser diffraction; and/or a FPF of the total dose less than 3.4 microns of about 25% or more. The dry powder is delivered to a patient, for example, with a capsule-based passive dry powder inhaler. The respirable dry particles may have a capsule emitted powder mass of at least 80% when emitted from a passive dry powder inhaler that has a resistance of about 0.036 sqrt(kPa)/liters per minute under the following conditions; an inhalation flow rate of 30 LPM for a period of 3 seconds using a size 3 capsule that contains a total mass of 10 mg, said total mass consisting of the respirable dry particles, and where the volume median geometric diameter of the respirable dry particles emitted from the inhaler as measured by laser diffraction is 5 microns or less.

Any one of the dry powders described herein may be used in a method for treating a fungal infection by administering an effective amount to the respiratory tract of a patient in need thereof; may be used for treating a fungal infection in a patient with asthma or cystic fibrosis by administering an effective amount to the respiratory tract of the asthma or cystic fibrosis patient in need thereof; may be used for treating aspergillosis by administering an effective amount to the respiratory tract of a patient in need thereof; may be used for treating allergic bronchopulmonary aspergillosis (ABPA) by administering an effective amount to the respiratory tract a patient in need thereof; may be used for treating or reducing the incidence or severity of an exacerbation, for example, an acute exacerbation caused by a fungal infection in the respiratory disease by administering an effective amount to the respiratory tract of a patient in need thereof; and/or may be used in treating a fungal infection in an immunocompromised patient by administering an effective amount to the respiratory tract of the immunocompromised patient in need thereof.

Any one of the dry powders described herein may be used to treat a fungal infection in an individual by administering to the respiratory tract of the individual an effective amount of the dry powder, where the fungal infection is treated.

Any one of the dry powders described herein may be used to treat a fungal infection in an individual with asthma or cystic fibrosis by administering to the respiratory tract of the individual an effective amount of the dry powder, where the fungal infection in the individual with asthma or cystic fibrosis patient.

Any one of the dry powders described herein may be used to treat aspergillosis in an individual, the use by administering to the respiratory tract of the individual an effective amount of the dry powder, where the aspergillosis is treated.

Any one of the dry powders described herein may be used to treat allergic bronchopulmonary aspergillosis (ABPA) in an individual, the use by administering to the respiratory tract of the individual an effective amount of the dry powder, where the ABPA is treated.

Any one of the dry powders described herein may be used to treat an exacerbation, for example, an acute exacerbation of the respiratory tract in an individual, the use by administering to the respiratory tract of the individual an effective amount of the dry powder, where the exacerbation, for example, an acute exacerbation is treated. In one example, the exacerbation, for example, an acute exacerbation is caused by a fungal infection.

Any one of the dry powders described herein may be used to treat a fungal infection of the respiratory tract in an immunocompromised by administering to the respiratory tract of the individual an effective amount of the dry powder, where the fungal infection is treated.

### BRIEF DESCRIPTION OF THE DRAWING

FIG. 1 is a survival plot for animals treated with placebo, 2 doses of Formulation I or an oral dose of Sporanox. The presented data indicate significantly increased survival following inhaled doses of Formulation I relative to both placebo and oral itraconazole.

FIG. 2: Cumulative mass dissolution of the ISM collected post-aerosolization of the itraconazole powder formulations from the RS01 at 60 L/min in the UniDose and then POD dissolution testing in a USP Apparatus II set-up.

FIG. 3: Cumulative percentage mass dissolution of the ISM collected post-aerosolization of the itraconazole suspension formulations from a Micro Mist nebulizer at 15 L/min in the UniDose and then POD dissolution testing in a USP Apparatus II set-up.

FIG. 4: Cumulative mass percent of the recovered dose from the different powder formulations of itraconazole deposited on stage 4 of the cNGI.

FIG. 5: Particle X-Ray Diffraction plot for Formulation I.

### DETAILED DESCRIPTION OF THE INVENTION

The invention relates to dry powder formulations of amorphous itraconazole. The dry powder formulations comprise, in one aspect, respirable dry particles that comprise amorphous itraconazole, sodium sulfate and optionally one or more other excipients. In such aspects, typically the amorphous itraconazole is about 45% to about 75%, the sodium sulfate is about 10% to about 55%, and the formulation optionally contains one or more other excipients, where all percentages are weight percentages on a dry basis and all the components of the respirable dry particles amount to 100%. Preferably, the other excipient is leucine, mannitol, or a combination of leucine and mannitol. Preferably, the amorphous itraconazole is about 45% to about 55%, or about 50%; and the sodium sulfate is about 30% to about 40%, or about 35%. If present, the one or more other excipients are preferably leucine, mannitol, or a combination of leucine and mannitol, and are about 5% to about 25%, or about 15%. In one aspect, a dry powder consists of respirable dry particles.

The dry powder formulations comprise, in another aspect, respirable dry particles that comprise amorphous itraconazole, sodium chloride and leucine and optionally one or more other excipients. In such aspects, typically the amorphous itraconazole is about 40% to about 60%, the sodium chloride is about 30% to about 50%, and the leucine is about 5% to about 25%, where all percentages are weight percentages on a dry basis and all the components of the respirable dry particles amount to 100%. Preferably, the amorphous itraconazole is about 45% to about 55%, or about 50%; the sodium chloride is about 35% to about 45%, or about 40%; and the leucine is about 5% to about 15%, or about 10%. In one aspect, a dry powder consists of respirable dry particles.

These dry powder formulations comprising respirable dry particles are small in geometric diameter, such as 10 microns or less or 5 microns or less, dense in mass (tap density) such as a tap density of 0.2 g/cc or greater, 0.3 g/cc or greater or greater than 0.4 g/cc, and dispersible in that they deagglomerate from each other with a relatively low amount of energy such as a 1/4 bar dispersibility ratio or a 0.5/4 bar dispersibility ratio of 1.5 or less, 1.4 or less or 1.3 or less.

The dry powder formulations of amorphous itraconazole may be administered to a patient by inhalation, such as oral inhalation. To achieve oral inhalation, a dry powder inhaler may be used, such as a passive dry powder inhaler. The dry powder formulations of amorphous itraconazole can be used to treat or prevent fungal infections in patients, such as aspergillosis infections. In some aspects, the dry powder formulations of amorphous itraconazole are used for acute treatment of fungal infections (e.g., moderate to high doses, e.g., 20-50 mg, 10-60 mg) for short-term dosing (e.g., one week, two weeks)). Alternatively, the amorphous itraconazole formulation may be used at a lower dose (e.g., 2 mg, 3 mg, 5 mg, or 10 mg nominal dose). Patients that would benefit from the dry powder formulations of amorphous itraconazole are, for example, those who suffer from asthma, cystic fibrosis, and/or who are at high risk of developing Aspergillus infections due to being severely immunocompromised, such as HIV patients and organ transplant patients. An inhaled formulation of amorphous itraconazole minimizes many of the downsides of oral or intravenous (IV) formulations of itraconazole in treating these patients. Existing oral formulations of itraconazole are limited by poor oral bioavailability,adverse side effects, and extensive drug-drug interactions. Pulmonary delivery of amorphous itraconazole overcomes these limitations and achieves high lung concentrations of drug, while minimizing systemic exposure and thus side effects. The amorphous itraconazole formulations of the present invention are improvements over existing formulations of itraconazole for several reasons, including but not limited to, rapid release and dissolution into the lung tissue including delivery to the deeper lung tissue. The amorphous itraconazole formulations may have rapid dissolution and permeation kinetics allowing for a quicker onset of treatment. This may be helpful during acute issues when speed of treatment onset is critical.

### Definitions

As used herein, the term “about” refers to a relative range of plus or minus 5% of a stated value, e.g., “about 20 mg” would be “20 mg plus or minus 1 mg”.

As used herein, the terms “administration” or “administering” of respirable dry particles refers to introducing respirable dry particles to the respiratory tract of a subject.

The term “capsule emitted powder mass” or “CEPM” as used herein refers to the amount of dry powder formulation emitted from a capsule or dose unit container during an inhalation maneuver. CEPM is measured gravimetrically, typically by weighing a capsule before and after the inhalation maneuver to determine the mass of powder formulation removed. CEPM can be expressed either as the mass of powder removed, in milligrams, or as a percentage of the initial filled powder mass in the capsule prior to the inhalation maneuver.

The term “dispersible” is a term of art that describes the characteristic of a dry powder or respirable dry particles to be dispelled into a respirable aerosol. Dispersibility of a dry powder or respirable dry particles is expressed herein, in one aspect, as the quotient of the volumetric median geometric diameter (VMGD) measured at a dispersion (i.e., regulator) pressure of 1 bar divided by the VMGD measured at a dispersion (i.e., regulator) pressure of 4 bar, or VMGD at 0.5 bar divided by the VMGD at 4 bar as measured by laser diffraction, such as with a HELOS/RODOS. These quotients are referred to herein as “1 bar/4 bar dispersibility ratio”, and “0.5 bar/4 bar dispersibility ratio”, respectively, and dispersibility correlates with a low quotient. For example, 1 bar/4 bar dispersibility ratio refers to the VMGD of a dry powder or respirable dry particles emitted from the orifice of a RODOS dry powder disperser (or equivalent technique) at about 1 bar, as measured by a HELOS or other laser diffraction system, divided by the VMGD of the same dry powder or respirable dry particles measured at 4 bar by HELOS/RODOS. Thus, a highly dispersible dry powder or respirable dry particles will have a 1 bar/4 bar dispersibility ratio or 0.5 bar/4 bar dispersibility ratio that is close to 1.0. Highly dispersible powders have a low tendency to agglomerate, aggregate or clump together and/or, if agglomerated, aggregated or clumped together, are easily dispersed or de-agglomerated as they emit from an inhaler and are breathed in by a subject. In another aspect, dispersibility is assessed by measuring the size emitted from an inhaler as a function of flowrate. As the flow rate through the inhaler decreases, the amount of energy in the airflow available to be transferred to the powder to disperse it decreases. A highly dispersible powder will have a size distribution such as is characterized aerodynamically by its mass median aerodynamic diameter (MMAD) or geometrically by its VMGD that does not substantially increase over a range of flow rates typical of inhalation by humans, such as about 15 to about 60 liters per minute (LPM), about 20 to about 60 LPM, or about 30 LPM to about 60 LPM. A highly dispersible powder will also have an emitted powder mass or dose, or a capsule emitted powder mass or dose, of about 80% or greater even at the lower inhalation flow rates. VMGD may also be called the volume median diameter (VMD), x50, or Dv50.

The term “dry particles” as used herein refers to respirable particles that may contain up to about 15% total of water and/or another solvent. Preferably the dry particles contain water and/or another solvent up to about 10% total, up to about 5% total, up to about I% total, or between 0.01% and 1% total, by weight of the dry particles, or can be substantially free of water and/or other solvent.

The term “dry powder” as used herein refers to compositions that comprise respirable dry particles. A dry powder may contain up to about 15% total of water and/or another solvent. Preferably the dry powder contain water and/or another solvent up to about 10% total, up to about 5% total, up to about 1% total, or between 0.01% and 1% total, by weight of the dry powder, or can be substantially free of water and/or other solvent. In one aspect, the dry powder is a respirable dry powder.

The term “effective amount,” as used herein, refers to the amount of agent needed to achieve the desired effect; such as treating a fungal infection, e.g., an Aspergillus infection, in the respiratory tract of a patient, e.g., an asthma patient, a Cystic Fibrosis (CF) patient, and an immunocompromised patient; treating allergic bronchopulmonary aspergillosis (ABPA); and treating or reducing the incidence or severity of an exacerbation, for example, an acute exacerbation of a respiratory disease. The actual effective amount for a particular use can vary according to the particular dry powder or respirable dry particle, the mode of administration, and the age, weight, general health of the subject, and severity of the symptoms or condition being treated. Suitable amounts of dry powders and dry particles to be administered, and dosage schedules for a particular patient can be determined by a clinician of ordinary skill based on these and other considerations.

As used herein, the term “emitted dose” or “ED” refers to an indication of the delivery of a drug formulation from a suitable inhaler device after a firing or dispersion event. More specifically, for dry powder formulations, the ED is a measure of the percentage of powder that is drawn out of a unit dose package and that exits the mouthpiece of an inhaler device. The ED is defined as the ratio of the dose delivered by an inhaler device to the nominal dose (i.e., the mass of powder per unit dose placed into a suitable inhaler device prior to firing). The ED is an experimentally-measured parameter, and can be determined using the method of USP Section 601 Aerosols, Metered-Dose Inhalers and Dry Powder Inhalers, Delivered-Dose Uniformity, Sampling the Delivered Dose from Dry Powder Inhalers, United States Pharmacopoeia convention, Rockville, Md., 13^{th} Revision, 222-225, 2007. This method utilizes an in vitro device set up to mimic patient dosing.

The terms “FPF (<X),” “FPF (<X microns),” and “fine particle fraction of less than X microns” as used herein, wherein X equals for example 3.4 microns, 4.4 microns, 5.0 microns, or 5.6 microns, refer to the fraction of a sample of dry particles that have an aerodynamic diameter of less than X microns. For example, FPF (A) can be determined by dividing the mass of respirable dry particles deposited on the stage two and on the final collection filter of a two-stage collapsed Andersen Cascade Impactor (ACI) by the mass of respirable dry particles weighed into a capsule for delivery to the instrument. This parameter may also be identified as “FPF_TD(<X),” where TD means total dose. A similar measurement can be conducted using an eight-stage ACI. An eight-stage ACI cutoffs are different at the standard 60 L/min flowrate, but the FPF_TD(<X) can be extrapolated from the eight-stage complete data set. The eight-stage ACI result can also be calculated by the USP method of using the dose collected in the ACI instead of what was in the capsule to determine FPF.

The terms “FPD (<X)”, “FPD<X microns”, FPD(<X microns)” and “fine particle dose of less than X microns” as used herein, wherein X equals for example 3.4 microns, 4.4 microns, 5.0 microns, or 5.6 microns, refer to the mass of a therapeutic agent delivered by respirable dry particles that have an aerodynamic diameter of less than X micrometers. FPD<X microns can be determined by using an eight-stage ACI at the standard 60 L/min flowrate and summing the mass deposited on the final collection filter, and either directly calculating or extrapolating the FPD value.

“Hausner ratio” is a term of art that refers to the tap density divided by the bulk density and typically correlates with bulk powder flowability (i.e., an increase in the Hausner ratio typically corresponds to a decrease in powder flowability.

The term “respirable” as used herein refers to dry particles or dry powders that are suitable for delivery to the respiratory tract (e.g., pulmonary delivery) in a subject by inhalation. Respirable dry powders or dry particles have a mass median aerodynamic diameter (MMAD) of less than about 10 microns, preferably about 5 microns or less.

As used herein, the term “respiratory tract” includes the upper respiratory tract (e.g., nasal passages, nasal cavity, throat, and pharynx), respiratory airways (e.g., larynx, trachea, bronchi, and bronchioles) and lungs (e.g., respiratory bronchioles, alveolar ducts, alveolar sacs, and alveoli).

The term “small” as used herein to describe respirable dry particles refers to particles that have a volume median geometric diameter (VMGD) of about 10 microns or less, preferably about 5 microns or less, or less than 5 microns.

As used herein “amorphous itraconazole” refers to an itraconazole formulation which exhibits a glass transition temperature (Tg). Preferably, the Tg is between 35° C. and 95° C., more preferably between 35° C. and 65° C. Alternatively, “amorphous itraconazole” refers to a formulation wherein the diffractogram generated by X-ray Powder Diffraction (XRPD) does not exhibit characteristic 20 values at ±0.20° of 8.78, 14.54 and 20.46.

### Dry Powders and Dry Particles

The invention relates to dry powder formulations comprising respirable thy particles comprising amorphous itraconazole, sodium sulfate and optionally one or more excipients. The amorphous itraconazole is about 45% to about 75%, the sodium sulfate is about 10% to about 55%, and the optional additional excipient, where all percentages are weight percentages on a dry basis and all the components of the respirable dry particles amount to 100%. Preferably, the amorphous itraconazole may be about 45% to about 55%, or about 50%. The sodium sulfate may be about 30% to about 40%, or about 35%. The optional additional excipient, when present, is about 5% to about 25%, or about 15%. The optional additional excipient, is preferably leucine, mannitol, or a combination of the two.

The respirable dry particles may contain about 1% to about 95% amorphous itraconazole by weight (wt %). It is preferred that the respirable dry particle contains an amount of amorphous itraconazole so that a therapeutically effective dose can be administered and maintained without the need to inhale large volumes of dry powder more than three time a day. For example, it is preferred that the respirable dry particles contain about 10% to 75%, about 15% to 75%, about 25% to 75%, about 30% to 70%, about 40% to 60%, about 20%, about 50%, or about 70% amorphous itraconazole by weight (wt %). The respirable dry particles may contain about 75%, about 80%, about 85%, about 90%, or about 95% amorphous itraconazole by weight (wt %). in particular embodiments, the range of amorphous itraconazole in the respirable dry particles is about 40% to about 90%, about 55% to about 85%, about 55% to about 75%. or about 65% to about 85%, by weight (wt %). The amount of amorphous itraconazole present in the respirable dry particles by weight is also referred to as the “drug load.”

The respirable dry particles can contain a total excipient content of about 10 wt % to about 99 wt %, with about 25 wt % to about 85 wt % , or about 40 wt % to about 55 wt % being more typical. The dry particles can contain a total excipient content of about 1 wt %, about 2 wt %, about 4 wt %, about 6 wt %, about 8 wt %, or less than about 10 wt %. In particular embodiments, the range is about 5% to about 50%, about 15% to about 50%, about 25% to about 50%, about 5% to about 40%, about 5% to about 30%, about 5% to about 20%, or about 5% to about 15%.

The invention also relates to dry powder formulations comprising respirable dry particles comprising amorphous itraconazole, sodium chloride and leucine. In such aspects, typically the amorphous itraconazole is about 40% to about 60%, the sodium chloride is about 30% to about 50%, and the leucine is about 5% to about 25%, where all percentages are weight percentages on a dry basis and all the components of the respirable dry particles amount to 100%. Preferably, the amorphous itraconazole is about 45% to about 55%, or about 50%; the sodium chloride is about 35% to about 45%, or about 40%; and the leucine is about 5% to about 15%, or about 10%.

These dry powder formulations comprising respirable dry particles are small in geometric diameter, such as 10 microns or less or 5 microns or less, dense in mass (tap density) such as a tap density of 0.2 g/cc or greater, 0.3 g/cc or greater or greater than 0.4 g/cc, and dispersible in that they deagglomerate from each other with a relatively low amount of energy such as a 1/4 bar dispersibility ratio or a 0.5/4 bar dispersihility ratio of 1.5 or less, 1.4 or less or 1.3 or less.

Preferred formulations are found in Table 1 below.

TABLE 1
Compositions

| Formulation | Composition (w/w), dry basis |
|---|---|
| I | 50.0% itraconazole (ITZ), 35.0% sodium sulfate, |
| | 15.0% leucine |
| II | 50.0% ITZ, 40.0% sodium chloride, 10.0% leucine |

Other Excipients

If desired, the respirable dry particles described herein can include another physiologically or pharmaceutically acceptable excipient in addition to sodium sulfate or sodium chloride. For example, a pharmaceutically-acceptable excipient includes monovalent and divalent metal cation salts, carbohydrates, sugar alcohols and amino acids.

### Salts

Suitable sodium salts that can be present in the respirable dry particles of the invention include, for example, sodium chloride, sodium citrate, sodium sulfate, sodium lactate, sodium acetate, sodium bicarbonate, sodium carbonate, sodium stearate, sodium ascorbate, sodium benzoate, sodium biphosphate, sodium phosphate, sodium bisulfite, sodium borate, sodium gluconate, sodium metasilicate and the like. In a preferred aspect, the dry powders and dry particles include sodium chloride, sodium citrate, sodium lactate, sodium sulfate, or any combination of these salts.

Suitable potassium salts include, for example, potassium chloride, potassium bromide, potassium iodide, potassium bicarbonate, potassium nitrite, potassium persulfate, potassium sulfite, potassium bisulfite, potassium phosphate, potassium acetate, potassium citrate, potassium glutamate, dipotassium guanylate, potassium gluconate, potassium malate, potassium ascorbate, potassium sorbate, potassium succinate, potassium sodium tartrate and any combination thereof.

Suitable magnesium salts include, for example, magnesium lactate, magnesium fluoride, magnesium chloride, magnesium bromide, magnesium iodide, magnesium phosphate, magnesium sulfate, magnesium sulfite, magnesium carbonate, magnesium oxide, magnesium nitrate, magnesium borate, magnesium acetate, magnesium citrate, magnesium gluconate, magnesium maleate, magnesium succinate, magnesium malate, magnesium taurate, magnesium orotate, magnesium glycinate, magnesium naphthenate, magnesium acetylacetonate, magnesium formate, magnesium hydroxide, magnesium stearate, magnesium hexafluorsilicate, magnesium salicylate or any combination thereof.

Suitable calcium salts include, for example, calcium chloride, calcium sulfate, calcium lactate, calcium citrate, calcium carbonate, calcium acetate, calcium phosphate, calcium alginate, calcium stearate, calcium sorbate, calcium gluconate and the like.

Preferred sodium salts are sodium sulfate and/or sodium chloride. A preferred magnesium salt is magnesium lactate.

### Carbohydrates and Sugar Alcohols

Carbohydrate excipients that are useful in this regard include the mono- and polysaccharides. Representative monosaccharides include dextrose (anhydrous and the monohydrate; also referred to as glucose and glucose monohydrate), galactose, mannitol, D-mannose, sorbose and the like. Representative disaccharides include lactose, maltose, sucrose, trehalose and the like. Representative trisaccharides include raffinose and the like. Other carbohydrate excipients include dextran, maltodextrin and cyclodextrins, such as 2-hydroxypropyl-beta-cyclodextrin can be used as desired. Representative sugar alcohols include mannitol, sorbitol and the like.

Preferred carbohydrates are mannitol, lactose, maltodextrin and trehalose.

### Carboxylate Moiety

Carboxylate moieties can be provided by carboxylic acids, salts thereof as well as by combinations of two or more carboxylic acids and or salts thereof. In a preferred embodiment, the carboxylate moiety is a hydrophilic carboxylic acid or salt thereof. Suitable carboxylic acids include but are not limited to hydroxydicarboxylic acids, hydroxytricarboxilic acids and the like. Citric acid and citrates, such as, for example sodium citrate, are preferred. Combinations or mixtures of carboxylic acids and or their salts also can be employed. The particles include a carboxylate moiety. In one embodiment of the invention, the carboxylate moiety includes at least two carboxyl groups.

### Amino Acids

Suitable amino acid excipients include any of the naturally occurring amino acids that form a powder under standard pharmaceutical processing techniques and include the non-polar (hydrophobic) amino acids and polar (uncharged, positively charged and negatively charged) amino acids, such amino acids are of pharmaceutical grade and are generally regarded as safe (GRAS) by the U.S. Food and Drug Administration. Representative examples of non-polar amino acids include alanine, isoleucine, leucine, trileucine, methionine, phenylalanine, proline, tryptophan and valine. Representative examples of polar, uncharged amino acids include cystine, glycine, glutamine, serine, threonine, and tyrosine. Representative examples of polar, positively charged amino acids include arginine, histidine and lysine. Representative examples of negatively charged amino acids include aspartic acid and glutamic acid.

Additionally, suitable amino acids also include non-naturally occurring arnion acids. Non-naturally occurring amino acids include, for example, beta-amino acids.

Further, suitable amino acids include hydrophobic amino acids, such as leucine, isoleucine, alanine, valine, phenylalanine, glycine and tryptophan.

Preferred amino acids are leucine and trileucine.

### Phospholipids

Suitable phospholipids include a phosphatidylcholine, phosphatidylethanolamine, phosphatidylglycerol, phosphatidylserine, phosphatidylinositol or a conhination thereof. In one embodiment, the phospholipids are endogenous to the lung. Specific examples of phospholipids are as follows: Dilaurvlolyphosphatidylcholine (C12;0) (DLPC), Dimyristoylphosphatidylcholine (C14;0) (DMPC), Dipalmitoylphosphatidylcholine (C16:0) (DPPC), Distearoylphosphatidylcholine (18:0) (DSPC), Dioleoylphosphatidylcholine (C18:1) (DOPC), Dilaurylotylphosphatidylglycerol (DLPG), Dimyristoylphosphatidylglycerol (DMPG). Dipalmitoylphosphatidylglycerol (DPPG), Distearoylphosphatidylglycerol (DSPC), Dioleoylphosphatidylglycerol (DOPG), Dimyristoyl phosphatidic acid (DMPA), Dipalmitoyl phosphatidic acid (DPPA), Dimyristoyl phosphatidylethanolamine (DMPE), Dipalmitoyl phosphatidylethanolamine (DPPE), Dimyristoyl phosphatidylserine (DMPS), Dipalmitoyl phosphatidylserine (DPPS), Dipalmitoyl sphingomyelin (DPSP), Distearoyl sphingomyelin (DSSP), or combinations thereof. Preferred phosphatidylcholines include DPPC and DSPC.

In one aspect, both a phospholipids and a multivalent salt such as a divalent salt are present. Preferred phospholipids include but are not limited to phosphatidic acid, phosphatidylcholines, phosphatidylethanolamines, phosphatidylglycerols, phosphatidylserines, phosphatidylinositols and combinations thereof. Preferred divalent salts include, for example chlorides of alkaline earth metals. Calcium chloride (CaCl₂) is most preferred. In another preferred embodiment, the multivalent salt is a pharmaceutically acceptable salt. In a preferred embodiment, the presence of a phospholipid and a multivalent salt combination increase the stability of the dry powder during storage than if the phospholipid had been present alone. For example, the combination increases the glass transition temperature to be greater than if the phospholipid had been present without the divalent salt.

In one aspect, the lipids formulations such as a phospholipid formulation forms a liposome.

### Surfactants

Suitable surfactant. As used herein, the term “surfactant” refers to any agent which preferentially absorbs to an interface between two immiscible phases, such as the interface between water and an organic polymer solution, a water/air interface or organic solvent/air interface. Surfactants generally possess a hydrophilic moiety and a lipophilic moiety, such that, upon absorbing to microparticles, they tend to present moieties to the external environment that do not attract similarly-coated particles, thus reducing particle agglomeration. Surfactants may also promote absorption of a therapeutic or diagnostic agent and increase bioavailability of the agent.

In addition to lung surfactants, such as, for example, phospholipids discussed above, suitable surfactants include but are not limited to hexadecanol; fatty alcohols such as polyethylene glycol (PEG); polyoxyethylene-9-lauryl ether; a surface active fatty acid, such as palmitic acid or oleic acid; glycocholate; surfactin; a poloxomer; a sorbitan fatty acid ester such as sorbitan trioleate (Span 85); tyloxapol; polysorbate such as polysorbate 20 and polysorbate 80; lecithin, soya lecithin, lauric acid palatine acid, stearic acid, erucic acid, behenic acid, or derivatives (such as esters and salts) thereof such as glyceryl behenate.

### Biodegradable Polymer, Copolymer, or Blend

Suitable polymers include a biocompatible, preferably biodegradable polymer, copolymer, or blend or other combination thereof. Useful polymers comprise polylactides, polylactide-glycolides, cyclodextrins, polyacrylates, methylcellulose, carboxymethylcellulose, polyvinyl alcohols, polyanhydrides, polylactams, polyvinyl pyrrolidones, polysaccharides (dextrans, starches, chitin, chitosan, etc.), hyaluronic acid, proteins (albumin, collagen, gelatin, etc.) and/or polyethylene glycol. Examples of polymeric resins that would be useful for the preparation of perforated ink microparticles include: styrene-butadiene, styrene-isoprene, styrene-acrylonitrile, ethylene-vinyl acetate, ethylene-acrylate, ethylene-acrylic acid, ethylene-methylacrylatate, ethylene-ethyl acrylate, vinyl-methyl methacrylate, acrylic acid-methyl methacrylate, and vinyl chloride-vinyl acetate. Those skilled in the art will appreciate that, by selecting the appropriate polymers, the delivery efficiency of the particulate compositions and/or the stability of the dispersions may be tailored to optimize the effectiveness of the active or agent.

The dry powders and/or respirable dry particles are preferably small, mass dense, and dispersible. To measure volumetric median geometric diameter (VMGD), a laser diffraction system may be used, e.g., a Spraytec system (particle size analysis instrument, Malvern Instruments) and a HELOS/RODOS system (laser diffraction sensor with dry dispensing unit, Sympatec GmbH). The respirable dry particles have a VMGD as measured by laser diffraction at the dispersion pressure setting (also called regulator pressure) of 1.0 bar at a maximum orifice ring pressure using a HELOS/RODOS system of about 10 microns or less, about 5 microns or less, about 4 μm or less, about 3 μm or less, about 1 μm to about 5 μm, about 1 μm to about 4 μm, about 1.5 μm to about 3.5 μm, about 2 μm to about 5 μm, about 2 μm to about 4 μm, or about 2 μm to about 3 μm. Preferably the VMGD is about 5 microns or less, or about 4 μm or less. In one aspect, the dry powders and/or respirable dry particles have a minimum VMGD of about 0.5 microns or about 1.0 micron.

The dry powders and/or respirable dry particles have 1 bar/4 bar dispersibility ratio and/or 0.5 bar/4 bar dispersibility ratio of less than about 2.0 (e.g., about 0.9 to less than about 2), about 1.7 or less (e.g., about 0.9 to about 1.7) about 1.5 or less (e.g., about 0.9 to about 1.5), about 1.4 or less (e.g., about 0.9 to about 1.4), or about 1.3 or less (e.g., about 0.9 to about 1.3), and preferably have a 1 bar/4 bar and/or a 0.5 bar/4 bar of about 1.5 or less (e.g., about 1.0 to about 1.5), and/or about 1.4 or less (e.g., about 1.0 to about 1.4).

In one aspect, the dry powders and/or respirable dry particles have a tap density of at least about 0.2 g/cm³, of at least about 0.25 g/cm³, a tap density of at least about 0.3 g/cm³, of at least about 0.35 g/cm³, a tap density of at least 0.4 g/cm³. In another aspect, the dry powders and/or respirable dry particles have a tap density of greater than 0.4 g/cm³ (e.g., greater than 0.4 g/cm³ to about 1.2 g/cm³), a tap density of at least about 0.45 g/cm³ e.g., about 0.45 g/cm³ to about 1.2 g/cm³), at least about 0.5 g/cm³ (e.g., about 0.5 g/cm³ to about 1.2 g/cm³), at least about 0.55 g/cm³ (e.g., about 0.55 g/cm³ to about 1.2 g/cm³), at least about 0.6 g/cm³ (e.g., about 0.6 g/cm³ to about 1.2 g/cm³), or at least about 0.6 g/cm³ to about 1.0 g/cm³. In another aspect, the dry powders and/or respirable dry particles have a tap density of about 0.2 g/cm³ to about 0.8 g/cm³.

The respirable dry particles, and the dry powders when the dry powders are respirable dry powders, have an MMAD of less than 10 microns, preferably an MMAD of about 5 microns or less, or about 4 microns or less. In one aspect, the respirable dry powders and/or respirable dry particles have a minimum MMAD of about 0.5 microns, or about 1.0 micron or about 2.0 microns.

The dry powders and/or respirable City particles have a FPF of less than about 5.6 microns (FPF<5.6 μm) of the total dose of at least about 35%, preferably at least about 45%, at least about 60%, between about 45% to about 80%, or between about 60% and about 80%.

The dry powders and/or respirable dry particles have a FPF of less than about 3.4 microns (FPF<3.4 μm) of the total dose of at least about 20%, preferably at least about 25%, at least about 30%, at least about 40%, between about 25% and about 60%, or between about 40% and about 60%.

The dry powders and/or respirable dry particles have a total water and/or solvent content of up to about 15% by weight, up to about 10% by weight, up to about 5% by weight, up to about 1%, or between about 0.01% and about 1%, or may be substantially free of water or other solvent.

The dry powders and/or respirable dry particles may be administered with love inhalation energy. In order to relate the dispersion of powder at different inhalation flow rates, volumes, and from inhalers of different resistances, the energy required to perform the inhalation maneuver may be calculated. Inhalation energy can be calculated from the equation E=R²Q²V where E is the inhalation energy in Joules, R is the inhaler resistance in kPa^{1/2}/LPM, Q is the steady flow rate in L/min and V is the inhaled air volume in L.

Healthy adult populations are predicted to be able to achieve inhalation energies ranging from 2.9 Joules for comfortable inhalations to 22 Joules for maximum inhalations by using values of peak inspiratory flow rate (PIFR) measured by Clarke et al. (Journal of Aerosol Med., 6(2), p. 99-110, 1993) for the flow rate Q from two inhaler resistances of 0.02 and 0.055 kPa^{1/2}/LPM, with an inhalation volume of 2 L based on both FDA guidance documents for dry powder inhalers and on the work of Tiddens et al. (Journal of Aerosol Med, 19(4), p. 456-465, 2006) who found adults averaging 2.2 L inhaled volume through a variety of DPIs.

Mild, moderate and severe adult COPD patients are predicted to be able to achieve maximum inhalation energies of 5.1 to 21 Joules, 5.2 to 19 Joules, and 2.3 to 18 Joules respectively. This is again based on using measured PIFR values for the flow rate Q in the equation for inhalation energy. The PIFR achievable for each group is a function of the inhaler resistance that is being inhaled through. The work of Broeders et al. (Eur Respir J, 18, p. 780-783, 2001) was used to predict maximum and minimum achievable PIFR through 2 dry powder inhalers of resistances 0.021 and 0.032 kPa^{1/2}/LPM for each.

Similarly, adult asthmatic patients are predicted to be able to achieve maximum inhalation energies of 7.4 to 21 Joules based on the same assumptions as the COPD population and PIFR data from Broeders et al.

Healthy adults and children, COPD patients, asthmatic patients ages 5 and above, and CF patients, for example, are capable of providing sufficient inhalation energy to empty and disperse the dry powder formulations of the invention.

The dry powders and/or respirable dry particles are characterized by a high emitted dose, such as a CEPM of at least 75%, at least 80%, at least 85%, at least 90%, at least 95%, from a passive dry powder inhaler subject to a total inhalation energy of about 5 Joules, about 3.5 Joules, about 2.4 Joules, about 2 Joules, about 1 Joule, about 0.8 Joules, about 0.5 Joules, or about 0.3 Joules is applied to the dry powder inhaler. The receptacle holding the dry powders and/or respirable dry particles may contain about 5 mg, about 7.5 mg, about 10 mg, about 15 mg, about 20 mg, or about 30 mg. In one aspect, the dry powders and/or respirable dry particles are characterized by a CEPM of 80% or greater and a VMGD of 5 microns or less when emitted from a passive dry powder inhaler having a resistance of about 0.036 sqrt(kPa)/liters per minute under the following conditions: an air flow rate of 30 LPM, ran for 3 seconds using a size 3 capsule that contains a total mass of 10 mg. In another aspect, the dry powders and/or respirable dry particles are characterized by a CEPM of 80% or greater and a VMGD of 5 microns or less when emitted from a passive dry powder inhaler having a resistance of about 0.036 sqrt(kPa)/liters per minute under the following conditions: an air flow rate of 20 LPM, ran for 3 seconds using a size 3 capsule that contains a total mass of 10 mg. In a further aspect, the dry powders and/or respirable dry particles are characterized by a CEPM of 80% or greater and a VMGD of 5 microns or less when emitted from a passive dry powder inhaler having a resistance of about 0.036 sqrt(kPa)/liters per minute under the following conditions: an air flow rate of 15 LPM, ran for 4 seconds using a size 3 capsule that contains a total mass of 10 mg.

The dry powder can fill the unit dose container, or the unit dose container can be at least 2% full, at least 5% full, at least 10% full, at least 20% full, at least 30% full, least 40% full, at least 50% full, at least 60% full, at least 70% full, at least 80% full, or at least 90% full. The unit dose container can be a capsule (e.g., size 000, 00, OE, 0, 1, 2, 3, and 4, with respective volumetric capacities of 1.37 ml, 950 μl, 770 μl, 680 μl, 480 μl, 360 μl, 270 μl, and 200 μl). The capsule is at least about 2% full, at least about 5% full, at least about 10% full, at least about 20% full, at least about 30% full, at least about 40% full, or at least about 50% full. The unit dose container can be a blister. The blister can be packaged as a single blister, or as part of a set of blisters, for example, 7 blisters, 14 blisters, 28 blisters, or 30 blisters. The one or more blister is preferably at least 30% full, at least 50% full, or at least 70% full.

An advantage of the invention is the production of powders that disperse well across a wide range of flow rates and are relatively flowrate independent. The dry powders and/or respirable dry particles of the invention enable the use of a simple, passive DPI for a wide patient population.

In particular aspects, the invention relates to a dry powders and/or respirable dry particles that comprise i) amorphous itraconazole, sodium sulfate and optionally one or more other excipients, or ii) amorphous itraconazole, sodium chloride and one or more other excipients. The dry powders and/or respirable dry particles are characterized by:
1. a VMGD at 1 bar as measured using a HELOS/RODOS system is about 10 microns or less, preferably about 5 microns or less;
2. a 1 bar/4 bar dispersibility ratio and/or a 0.5 bar/4 tsar dispersibility ratio of about 1.5 or less, about 1.4 or less, or about 1.3 or less;
3. a tap density of about 0.2 g/cm³ or greater, about 0.3 g/cm³ or greater, about 0.4 g/cm³ or greater, greater than 0.4 g/cm³, about 0.45 g/cm³ or greater, or about 0.5 g/cm³ or greater;
4. a MMAD of about 10 microns or less, preferably about 5 microns or less;
5. a FPF<5.6 of the total dose of at least about 45%, or at least about 60%; and/or
6. a FPF<3.4 of the total dose of at least about 25%, or at least about 40%.

The dry powders and/or respirable thy particles described by any of the ranges or specifically disclosed formulations, characterized in the previous paragraph, may be filled into a receptacle, for example a capsule or a blister. When the receptacle is a capsule, the capsule is, for example, a size 2 or a size 3 capsule, and is preferably a size 3 capsule. The capsule material may be, for example, gelatin or HPMC (Hydroxypropyl methylcellulose), and is preferably HPMC.

The dry powder and/or respirable dry particles described and characterized above may be contained in a dry powder inhaler (DPI). The DPI may be a capsule-based DPI or a blister-based DPI, and is preferably a capsule-based DPI. More preferably, the dry powder inhaler is selected from the RS01 family of dry powder inhalers (Plastiape S.p.A., Italy). More preferably, the dry powder inhaler is selected from the RS01 HR or the RS01 UHR2, Most preferably, the dry powder inhaler is the RS01 HR.

### Methods for Preparing Dry Powders and Dry Particles

The respirable dry particles and dry powders can be prepared using any suitable method. Many suitable methods for preparing dry powders and/or respirable dry particles are conventional in the art, and include single and double emulsion solvent evaporation, spray drying, spray-freeze drying, milling (e.g., jet milling), blending, solvent extraction, solvent evaporation, phase separation, simple and complex coacervation, interfacial polymerization, suitable methods that involve the use of supercritical carbon dioxide (CO₂), sonocrystallization, nanoparticle aggregate formation and other suitable methods, including combinations thereof. Respirable dry particles can be made using methods for making microspheres or microcapsules known in the art. These methods can be employed under conditions that result in the formation of respirable dry particles with desired aerodynamic properties (e.g., aerodynamic diameter and geometric diameter). If desired, respirable dry particles with desired properties, such as size and density, can be selected using suitable methods, such as sieving.

Suitable methods for selecting respirable dry particles with desired properties, such as size and density, include wet sieving, dry sieving, and aerodynamic classifiers (such as cyclones).

The respirable dry particles are preferably spray dried. Suitable spray-drying techniques are described, for example, by K. Masters in “Spray Drying Handbook”, John Wiley & Sons, New York (1984). Generally, during spray-drying, heat from a hot gas such as heated air or nitrogen is used to evaporate a solvent from droplets formed by atomizing a continuous liquid feed. When hot air is used, the moisture in the air is at least partially removed before its use. When nitrogen is used, the nitrogen gas can be run “dry”, meaning that no additional water vapor is combined with the gas. If desired the moisture level of the nitrogen or air can be set before the beginning of spray dry run at a fixed value above “dry” nitrogen. If desired, the spray drying or other instruments, e.g., jet milling instrument, used to prepare the dry particles can include an inline geometric particle sizer that determines a geometric diameter of the respirable dry particles as they are being produced, and/or an inline aerodynamic particle sizer that determines the aerodynamic diameter of the respirable dry particles as they are being produced.

For spray drying, solutions, emulsions or suspensions that contain the components of the dry particles to be produced in a suitable solvent (e.g., aqueous solvent, organic solvent, aqueous-organic mixture or emulsion) are distributed to a drying vessel via an atomization device. For example, a nozzle or a rotary atomizer may be used to distribute the solution or suspension to the drying vessel. The nozzle can be a two-fluid nozzle, which is in an internal mixing setup or an external mixing setup. Alternatively, a rotary atomizer having a 4- or 24-vaned wheel may be used. Examples of suitable spray dryers that can be outfitted with a rotary atomizer and/or a nozzle, include, a Mobile Minor Spray Dryer or the Model PSD-1, both manufactured by GEA Niro, Inc. (Denmark), Büchi B-290 Mini Spray Dryer (BÜCHI Labortechnik AG, Flawil, Switzerland), ProCepT Formatrix R&D spray dryer (ProCepT nv, Zelzate, Belgium), among several other spray dryer options. Actual spray drying conditions will vary depending, in part, on the composition of the spray drying solution, suspension or emulsion and material flow rates. The person of ordinary skill will be able to determine appropriate conditions based on the compositions of the solution, emulsion or suspension to be spray dried, the desired particle properties and other factors. In general, the inlet temperature to the spray dryer is about 90° C. to about 300° C. The spray dryer outlet temperature will vary depending upon such factors as the feed temperature and the properties of the materials being dried. Generally, the outlet temperature is about 50° C. to about 150° C. If desired, the respirable dry particles that are produced can be fractionated by volumetric size, for example, using a sieve, or fractioned by aerodynamic size, for example, using a cyclone, and/or further separated according to density using techniques known to those of skill in the art.

To prepare the respirable dry particles of the invention, generally, a solution, emulsion or suspension that contains the desired components of the dry powder (i.e., a feed stock) is prepared and spray dried under suitable conditions. Preferably, the dissolved or suspended solids concentration in the feed stock is at least about 1 g/L, at least about 2 g/L, at least about 5 g/L, at least about 10 g/L, at least about 15 g/L, at least about 20 g/L, at least about 30 g/L, at least about 40 g/L, at least about 50 g/L, at least about 60 g/L, at least about 70 g/L, at least about 80 g/L, at least about 90 g/L, or at least about 100 g/L. The feed stock can be provided by preparing a single solution, suspension or emulsion by dissolving, suspending, or emulsifying suitable components (e.g., salts, excipients, other active ingredients) in a suitable solvent. The solution, emulsion or suspension can be prepared using any suitable methods, such as bulk mixing of dry and/or liquid components or static mixing of liquid components to form a combination. For example, a hydrophilic component (e.g., an aqueous solution) and a hydrophobic component (e.g., an organic solution) can be combined using a static mixer to form a combination. The combination can then be atomized to produce droplets, which are dried to form respirable dry particles. Preferably, the atomizing step is performed immediately after the components are combined in the static mixer. Alternatively, the atomizing step is performed on a bulk mixed solution.

The feed stock, or components of the feed stock, can be prepared using any suitable solvent, such as an organic solvent, an aqueous solvent or mixtures thereof. Suitable organic solvents that can be employed include but are not limited to alcohols such as, for example, ethanol, methanol, propanol, isopropanol, butanols, and others. Other organic solvents include but are not limited to tetrahydrofuran (THF), perfluorocarbons, dichloromethane, chloroform, ether, ethyl acetate, methyl tert-butyl ether and others. Co-solvents that can be employed include an aqueous solvent and an organic solvent, such as, but not limited to, the organic solvents as described above. Aqueous solvents include water and buffered solutions. Preferred solvents include water and tetrahydrofuran (THF).

Various methods (e.g., static mixing, bulk mixing) can be used for mixing the solutes and solvents to prepare feedstocks, which are known in the art. If desired, other suitable methods of mixing may be used. For example, additional components that cause or facilitate the mixing can be included in the feedstock. For example, carbon dioxide produces fizzing or effervescence and thus can serve to promote physical mixing of the solute and sol vents.

The feed stock or components of the feed stock can have any desired pH, viscosity or other properties. If desired, a pH buffer can be added to the solvent or co-solvent or to the formed mixture. Generally, the pH of the mixture ranges from about 3 to about 8.

Dry powder and/or respirable dry particles can be fabricated and then separated, for example, by filtration or centrifugation by means of a cyclone, to provide a particle sample with a preselected size distribution. For example, greater than about 30%, greater than about 40%, greater than about 50%, greater than about 60%, greater than about 70%, greater than about 80%, or greater than about 90% of the respirable dry particles in a sample can have a diameter within a selected range. The selected range within which a certain percentage of the respirable dry particles fall can be, for example, any of the size ranges described herein, such as between about 0.1 to about 3 microns VMGD.

Methods of analyzing the dry powders and/or respirable dry particles is found in the Exemplification section below.

### Therapeutic Use and Methods

The dry powders and/or respirable dry particles of the present invention are suitable for administration to the respiratory tract, for example to a subject in need thereof for the treatment of respiratory (e.g., pulmonary) diseases, such as asthma, especially severe asthma, cystic fibrosis, and severely immunocompromised patients. This treatment is especially useful in treating aspergillosis infections.

In other aspects, the invention is a method for the treatment, reduction in incidence or severity, or prevention of exacerbations, for example, an acute exacerbations caused by a fungal infection in the respiratory tract, such as an aspergillosis infection.

In other aspects, the invention is a method for relieving the symptoms of a respiratory disease and/or a chronic pulmonary disease, such as asthma, especially severe asthma, cystic fibrosis, and severely immunocompromised patients.

In other aspects, the invention is a method for improving lung function of a patient with a respiratory disease and/or a chronic pulmonary disease, such as such as asthma, especially severe asthma, cystic fibrosis, and severely immunocompromised patients.

In one aspect, the invention relates to a method of treating respiratory (e.g., pulmonary) diseases, such as cystic fibrosis, asthma, especially severe asthma, and severely immunocompromised patients, the method comprising administering dry powders and/or respirable dry particles to the respiratory tract of a subject in need thereof, thereby treating respiratory (e.g., pulmonary) diseases, such as cystic fibrosis, asthma, especially severe asthma, and severely immunocompromised patients. This treatment is especially useful in treating Aspergillus infections e.g., Aspergillus fumigatus infections). This treatment is also useful for treating fungal infections sensitive to itraconazole. Another aspect of the invention is treating allergic bronchopulmonary aspergillosis (ABPA), for example, in patients with pulmonary disease such as asthma or cystic fibrosis. The invention may also allow for treating an individual with a resistant fungal infection by administering an inhaled anti-fungal formulation. In a further aspect, the invention is a method for prophylaxis or treatment of invasive fungal infections in an immunocompromised patient population.

In some aspects, about 2 mg, about 3 mg, about 4 mg, 5 mg, about 10 mg, about 15 mg, about 20 mg, about 25 mg, about 30 mg, about 35 mg, about 40 mg, about 50 mg, about 2 mg to about 35 mg, about 5 mg to about 50 mg, about 10 mg to about 50 mg, about 10 mg to about 60 mg, about 15 mg to about 50 mg, or about 20 mg to about 50 mg, nominal doses may be administered.

The dry powder formulation may be administered in one or more doses to achieve a lung concentration of about 500 ng/mL, about 800 ng/mL, about 1200 ng/mL, about 1600 ng/mL, about 2000 ng/mL, about 3000 ng/mL, about 4000 ng/mL, about 5000 ng/mL, about 5000 ng/mL, about 6000 ng/mL, about 7000 ng/mL, about 8000 ng/mL, between 2000 ng/mL to 8000 ng/mL, about 2000 ng/mL to 8100 ng/mL, or about 2000 ng/mL to 10000 ng/mL. The lung concentration may be measured at the maximum concentration (i.e., the “Cₘₐₓ”) of the anti-fungal agent in the lung tissue, or at any point in time. The lung concentration may be measured at any point in the dosing cycle and may be calculated before or at steady state.

The dry powder formulation may be administered once a day, twice a day, once every other day, or once every three days for approximately 7 days, 14 days, 21 days, 28 days, or 1 month. In some embodiments, one or more doses needed to achieve a fungicidal concentration of amorphous itraconazole in the lung is administered daily.

In another aspect of the invention, a fungicidal dose of dry powders and/or respirable dry particles may be administered to the respiratory tract of a subject in need thereof, thereby treating respiratory (e.g., pulmonary) diseases, such as cystic fibrosis, asthma, especially severe asthma, and severely immunocompromised patients. The fungicidal dose needed to achieve a minimum fungicidal concentration (MFC) (e.g., MFC50, MFC90) will vary depending on the specific fungus causing the infection, but may be 0.05 mg/L to about 16 mg/L. Various methods and assays for determining lung and plasma concentrations are known in the art and may be used to measure the lung and plasma concentrations during and after administration of the amorphous itraconazole dry powders. For example, bioassays or high performing HPLC may be used to measure the amount of anti-fungal active agent in the lung (e.g., using induced sputum, bronchial lavage, spontaneous sputum) after the patient has been taking the drug for at least 7 days, at least 14 days, at least 21 days, or at least 28 days.

In other aspects, the invention is a method for the treatment, reduction in incidence or severity, or prevention of acute exacerbations caused by a fungal infection in the respiratory tract, such as an Aspergillus infection. In another aspect, the invention is a method for the treatment, reduction in incidence or severity, or prevention of exacerbations caused by a fungal infection in the respiratory tract, such as an Aspergillus infection. In another aspect, the invention is a method for the treatment, reduction in incidence or severity, or prevention of exacerbations caused by allergic bronchopulmonary aspergillosis (ABPA), for example, in patients with pulmonary disease such as asthma or cystic fibrosis. In a further aspect, the invention is a method for prophylaxis or treatment of invasive fungal infections in an immunocompromised patient population.

In other aspects, the invention is a method for relieving the symptoms of a respiratory disease and/or a chronic pulmonary disease, such as cystic fibrosis, asthma, especially severe asthma and severely immunocompromised patients. In another aspect, the invention is a method for relieving the symptoms of allergic bronchopulmonary aspergillosis (ABPA) in these patient populations. In yet another aspect, the invention is a method for reducing inflammation, sparing the use of steroids, or reducing the need for steroidal treatment.

In other aspects, the invention is a method for improving lung function of a patient with a respiratory disease and/or a chronic pulmonary disease, such as such as cystic fibrosis, asthma, especially severe asthma and severely immunocompromised patients. In another aspect, the invention is a method for improving lung function of a patient with allergic bronchopulmonary aspergillosis (ABPA).

The dry powders and/or respirable dry particles can be administered to the respiratory tract of a subject in need thereof using any suitable method, such as instillation techniques, and/or an inhalation device, such as a dry powder inhaler (DPI) or metered dose inhaler (MDI). A number of DPIs are available, such as, the inhalers disclosed is U.S. Pat. Nos. 4,995,385 and 4,069,819, Spinhaler® (Fisons, Loughborough, U.K.), Rotahalers®, Diskhaler® and Diskus® (GlaxoSmithKline, Research Triangle Technology Park, North Carolina), FlowCaps® (Hovione, Loures, Portugal), Inhalators® (Boehringer-Ingelheim, Germany), Aerolizer® (Novartis, Switzerland), high-resistance, ultrahigh-resistance and low-resistance RS-01 (Plastiape, Italy) and others known to those skilled in the art.

The following scientific journal articles are incorporated by reference for their thorough overview of the following dry powder inhaler (DPI) configurations: 1) Single-dose Capsule DPI, 2) Multi-dose Blister DPI, and 3) Multi-dose Reservoir DPI. N. Islam, E. Gladki, “Dry powder inhalers (DPIs)—A review of device reliability and innovation”, International Journal of Pharmaceuticals, 360(2008):1-11, H. Chystyn, “Diskus Review”, International Journal of Clinical Practice, June 2007, 61, 6, 1022-1036. H. Steckel, B. Muller, “In vitro evaluation of dry powder inhalers I: drug deposition of commonly used devices”, International Journal of Pharmaceuticals, 154(1997):19-29. Some representative capsule-based DPI units are RS-01 (Plastiape, Italy), Turbospin® (PH&T, Brezhaler® (Novartis, Switzerland), Aerolizer (Novartis, Switzerland), Podhaler® (Novartis, Switzerland), HandiHaler® (Boehringer Ingelheim, Germany), (Civitas, Massachusetts), Dose One® (Dose One, Maine), and Eclipse® (Rhone Poulenc Rorer). Some representative unit dose DPIs are Conix® (3M, Minnesota), Cricket® (Mannkind, California), Dreamboat® (Mannkind, California), Occoris® (Team Consulting, Cambridge, UK), Solis® (Sandoz), Trivair® (Trimel Biopharma, Canada), Twincaps® (Hovione, Loures, Portugal). Some representative blister-based DPI units are Diskus® (GlaxoSmithKline (GSK), UK), Diskhaler® (GSK), Taper Dry® (3M, Minnesota), (GSK), Twincer® (University of Groningen, Netherlands), Aspirair® (Vectura, UK), Acu-Breathe® (Respirics, Minnesota, USA), Exubra® (Novartis, Switzerland), Gyrohaler® (Vectura, UK), Omnihaler® (Vectura, UK), Microdose® (Microdose Therapeutix, USA), Multihaler® (Cipla, India) Prohaler® (Aptar), Technohaler® (Vectura, UK), and Xcelovair® (Mylan, Pa.). Some representative reservoir-based DPI units are Clickhaler® (Vectura), Next DPI® (Chiesi), Easyhaler® (Orion), Novolizer® (Meda), Pulmojet® (sanofi-aventis), (Chiesi), Skyehaler® (Skyepharma), Duohaler® (Vectura), Taifun® (Akela), Flexhaler® (AstraZeneca, Sweden), Turbuhaler® (AstraZeneca, Sweden), and Twisthaler® (Merck), and others known to those skilled in the art.

Generally, inhalation devices (e.g., DPIs) are able to deliver a maximum amount of dry powder or dry particles in a single inhalation, which is related to the capacity of the blisters, capsules (e.g. size 000, 00, 0E, 0, 1, 2, 3, and 4, with respective volumetric capacities of 1.37 ml, 950 μl, 770 μl, 680 μl, 480 μl, 360 μl, 270 μl, and 200 μl) or other means that contain the dry powders and/or respirable dry particles within the inhaler. Preferably, the blister has a volume of about 360 microliters or less, about 270 microliters or less, or more preferably, about 200 microliters or less, about 150 microliters or less, or about 100 microliters or less. Preferably, the capsule is a size 2 capsule, or a size 4 capsule. More preferably, the capsule is a size 3 capsule. Accordingly, delivery of a desired dose or effective amount may require two or more inhalations. Preferably, each dose that is administered to a subject in need thereof contains an effective amount of respirable dry particles or dry powder and is administered using no more than about 4 inhalations. For example, each dose of dry powder or respirable dry particles can be administered in a single inhalation or 2, 3, or 4 inhalations. The dry powders and/or respirable dry particles are preferably administered in a single, breath-activated step using a passive DPI. When this type of device is used, the energy of the subject's inhalation both disperses the respirable dry particles and draws them into the respiratory tract.

The dry powder can include blends of the dry particles with lactose, such as large lactose carrier particles that are greater than 10 microns, 20 microns to 500 microns, and preferably between 25 microns and 250 microns.

Dry powders and/or respirable dry particles suitable for use in the methods of the invention can travel through the upper airways (i.e., the oropharynx and larynx), the lower airways, which include the trachea followed by bifurcations into the bronchi and bronchioli, and through the terminal bronchioli which in turn divide into respiratory bronchioli leading then to the ultimate respiratory zone, the alveoli or the deep lung. In one embodiment of the invention, most of the mass of respirable dry particles deposit in the deep lung. In another embodiment of the invention, delivery is primarily to the central airways. In another embodiment, delivery is to the upper airways. In a preferred embodiment, most of the mass of the respirable dry particles deposit in the conducting airways.

If desired or indicated, the dry powders and respirable dry particles described herein can be administered with one or more other therapeutic agents. The other therapeutic agents can be administered by any suitable route, such as orally, parenterally intravenous, intra-arterial, intramuscular, or subcutaneous injection), topically, by inhalation (e.g., intrabronchial, intranasal or oral inhalation, intranasal drops), rectally, vaginally, and the like. The respirable dry particles and dry powders can be administered before, substantially concurrently with, or subsequent to administration of the other therapeutic agent. Preferably, the dry powders and/or respirable dry particles and the other therapeutic agent are administered so as to provide substantial overlap of their pharmacologic activities.

### Exemplification

Materials used in the following Examples and their sources are listed below. Sodium sulfate, sodium chloride, L-leucine, mannitol, and tetrahydrofuran were obtained from Sigma-Aldrich Co. (St. Louis, Mo.), Spectrum Chemicals (Gardena, Calif.), or Merck (Darmstadt, Germany). Itraconazole was obtained from Neuland Laboratories Limited (Telangana, India). Ultrapure (Type II ASTM) water was from a water purification system (Millipore Corp., Billerica, Mass.), or equivalent.

Methods:

Geometric or Volume Diameter. Volume median diameter (x50 or Dv50), which may also be referred to as volume median geometric diameter (VMGD), was determined using a laser diffraction technique. The equipment consisted of a HELOS diffractometer and a RODOS dry powder disperser (Sympatec, Inc., Princeton, N.J.). The RODOS disperser applies a shear force to a sample of particles, controlled by the regulator pressure (typically set at 1.0 bar with maximum orifice ring pressure) of the incoming compressed dry air. The pressure settings may be varied to vary the amount of energy used to disperse the powder. For example, the dispersion energy may be modulated by changing the regulator pressure from 0.2 bar to 4.0 bar. Powder sample is dispensed from a microspatula into the RODOS funnel. The dispersed particles travel through a laser beam where the resulting diffracted light pattern produced is collected, typically using an R1 lens, by a series of detectors. The ensemble diffraction pattern is then translated into a volume-based particle size distribution using the Fraunhofer diffraction model, on the basis that smaller particles diffract light at larger angles. The HELOS/RODOS instrument was run in controlled environmental conditions of 18 to 25° C. and relative humidity (RH) between 25 and 35%. Using this method, geometric standard deviation (GSD) for the volume diameter was also determined.

Volume median diameter can also be measured using a method where the powder is emitted from a dry powder inhaler device. The equipment consisted of a Spraytec laser diffraction particle size system (Malvern, Worcestershire, UK), “Spraytec”. Powder formulations were filled into size 3 HPMC capsules (Capsugel V-Caps) by hand with the fill weight measured gravimetrically using an analytical balance (Mettler Tolerdo XS205, Columbus, Ohio). A capsule based passive dry powder inhaler (RS01 Model 7, High resistance Plastiape S.p.A., Italy) was used which had a specific resistance of 0.036 kPa^{1/2}LPM⁻¹. Flow rate and inhaled volume were set using a timer controlled solenoid valve with flow control valve (TPK2000, Copley Scientific, UK). Capsules were placed in the dry powder inhaler, punctured and the inhaler sealed inside a cylinder. The cylinder was connected to a positive pressure air source with steady air flow through the system measured with a mass flow meter and its duration controlled with a timer controlled solenoid valve. The exit of the dry powder inhaler was exposed to room pressure and the resulting aerosol)et passed through the laser of the diffraction particle sizer (Spraytec) in its open bench configuration before being captured by a vacuum extractor. The steady air flow rate through the system was initiated using the solenoid valve and the particle size distribution was measured via the Spraytec at 1 kHz for the duration of the single inhalation maneuver with a minimum of 2 seconds. The Spraytec instrument was run in controlled environmental conditions of 18 to 25° C. and relative humidity (RH) between 25 and 35%. Particle size distribution parameters calculated included the volume median diameter (Dv50) and the geometric standard deviation (GSD) and the fine particle fraction (FPF) of particles less than 5 micrometers in diameter. At the completion of the inhalation duration, the dry powder inhaler was opened, the capsule removed and re-weighed to calculate the mass of powder that had been emitted from the capsule during the inhalation duration (capsule emitted powder mass or CEPM).

Fine Particle Fraction, The aerodynamic properties of the powders dispersed from an inhaler device were assessed with an Mk-II 1 ACFM Andersen Cascade Impactor (Copley Scientific Limited, Nottingham, UK) (ACI) or a Next Generation Impactor (Copley Scientific Limited, Nottingham, UK) (NGI). The ACI instrument was run in controlled environmental conditions of 18 to 25° C. and relative humidity (RH) between 25 and 35%. The instrument consists of eight stages that separate aerosol particles based on inertial impaction. At each stage, the aerosol stream passes through a set of nozzles and impinges on a corresponding impaction plate. Particles having small enough inertia will continue with the aerosol stream to the next stage, while the remaining particles will impact upon the plate. At each successive stage, the aerosol passes through nozzles at a higher velocity and aerodynamically smaller particles are collected on the plate. After the aerosol passes through the final stage, a filter collects the smallest particles that remain, called the “final collection filter”. Gravimetric and/or chemical analyses can then be performed to determine the particle size distribution. A short stack cascade impactor, also referred to as a collapsed cascade impactor, is also utilized to allow for reduced labor time to evaluate two aerodynamic particle size cut-points. With this collapsed cascade impactor, stages are eliminated except those required to establish fine and coarse particle fractions.

The impaction techniques utilized allowed for the collection of two or eight separate powder fractions. The capsules (HPMC, Size 3; Capsugel Vcaps, Morristown, N.J.) were hand filled with powder to a specific weight and placed in a hand-held, breath-activated dry powder inhaler (DPI) device, the RS01 DPI high resistance (Plastiape, Osnago, Italy). The capsule was punctured and the powder was drawn through the cascade impactor operated at a flow rate of 60.0 L/min for 2.0 s. At this flowrate, the calibrated cut-off diameters for the eight stages are 8.6, 6.5. 4.4, 3.3, 2.0, 1.1, 0.5 and 0.3 microns and for the two stages used with the short stack cascade impactor, based on the Andersen Cascade Impactor, the cut-off diameters are 5.6 microns and 3.4 microns. The fractions were collected by placing filters in the apparatus and determining the amount of powder that impinged on them by gravimetric measurements or chemical measurements on an HPLC. The fine particle fraction of the total dose of powder (FPF_{TD}) less than or equal to an effective cut-off aerodynamic diameter was calculated by dividing the powder mass recovered from the desired stages of the impactor by the total particle mass in the capsule. Results are reported for the eight-stage normal stack cascade impactor as the fine particle fraction of less than 4.4 microns (FPF_{TD}<4.4 microns) and the fine particle fraction of less than 2.0 microns (FPF_{TD}<2.0 microns), and the two-stage short stack cascade impactor as the fine particle fraction of less than 5.6 microns (FPF_{TD}<5.6 microns) and the fine particle fraction of less than 3.4 microns (FPF_{TD}<3.4 microns). The fine particle fraction can alternatively be calculated relative to the recovered or emitted dose of powder by dividing the powder mass recovered from the desired stages of the impactor by the total powder mass recovered in the impactor.

Similarly, for FPF measurements utilizing the NGI, the NGI instrument was run in controlled environmental conditions of 18 to 25° C. and relative humidity (RH) between 25 and 35%. The instrument consists of seven stages that separate aerosol particles based on inertial impaction and can be operated at a variety of air flow rates. At each stage, the aerosol stream passes through a set of nozzles and impinges on a corresponding impaction surface. Particles having small enough inertia will continue with the aerosol stream to the next stage, while the remaining particles will impact upon the surface. At each successive stage, the aerosol passes through nozzles at a higher velocity and aerodynamically smaller particles are collected on the plate. After the aerosol passes through the final stage, a micro-orifice collector collects the smallest particles that remain. Chemical analyses can then be performed to determine the particle size distribution. The capsules (HPMC, Size 3; Capsugel) were hand filled with powder to a specific weight and placed in a hand-held, breath-activated dry powder inhaler (DPI) device, the high resistance RS01 DPI (Plastiape). The capsule was punctured and the powder was drawn through the cascade impactor operated at a specified flow rate for 2.0 Liters of inhaled air. At the specified flow rate, the cut-off diameters for the stages were calculated. The fractions were collected by placing wetted filters in the apparatus and determining the amount of powder that impinged on them by chemical measurements on an HPLC. The fine particle fraction of the total dose of powder (FPF_{TD}) less than or equal to an effective cut-off aerodynamic diameter was calculated by dividing the powder mass recovered from the desired stages of the impactor by the total particle mass in the capsule. Results are reported for the NGI as the fine particle fraction of less than 5.0 microns (FPF_{TD}<5.0 microns)

Aerodynamic Diameter. Mass median aerodynamic diameter (MMAD) was determined using the information obtained by the Andersen Cascade Impactor (ACI). The cumulative mass under the stage cut-off diameter is calculated for each stage and normalized by the recovered dose of powder. The MMAD of the powder is then calculated by linear interpolation of the stage cut-off diameters that bracket the 50th percentile. An alternative method of measuring the MMAD is with the Next Generation Pharmaceutical Impactor (NGI). Like the ACI, the MMAD is calculated with the cumulative mass under the stage cut-off diameter is calculated for each stage and normalized by the recovered dose of powder. The MMAD of the powder is then calculated by linear interpolation of the stage cut-off diameters that bracket the 50th percentile.

Fine Particle Dose. The fine particle dose (FPD) is determined using the information obtained from the ACI. Alternatively, the FPD is determined using the information obtained from the NGI. The fine particle dose indicates the mass of one or more therapeutics in a specific size range and can be used to predict the mass which will reach a certain region in the respiratory tract. The fine particle dose can be measured gravimetrically or chemically. If measured gravimetrically, since the dry particles are assumed to be homogenous, the mass of the powder on each stage and collection filter can be multiplied by the fraction of therapeutic agent in the formulation to determine the mass of therapeutic. If measured chemically, the powder from each stage or filter is collected, separated, and assayed for example on an HPLC to determine the content of the therapeutic. The cumulative mass deposited on the final collection filter, and stages 6, 5, 4, 3, and 2 for a single dose of powder, contained in one or more capsules, actuated into the ACI is equal to the fine particle dose less than 5.0 microns (FPD<5.0 microns). The cumulative mass deposited on the final collection filter, and stages 6, 5 and 4 for a single dose of powder, contained in one or more capsules, actuated into the ACI is equal to the fine particle dose less than 2.0 microns (FPD<2.0 microns). The quotient of these two values is expressed as FPD<2.0 μm/FPD<5.0 fun. The higher the ratio, the higher the percentage of therapeutic that enters the lungs which is expected to penetrate to the alveolar regions of the lung. The lower the ratio, the lower the percentage of therapeutic that enters the lungs, which is expected to penetrate to the alveolar regions of the lung. For some therapies that target the central or conducting airways, a lower ratio such as less than 40%, less than 30%, or less than 20% is desired. For other therapies that target the deep lung, a higher ratio such as 40% or greater, 50% or greater, or 60% or greater is desired. Similarly, for FPD measurements utilizing the NGI, the NGI instrument was run as described in the Fine Particle Fraction description in the Exemplification section. The cumulative mass deposited on each of the stages at the specified flow rate is calculated and the cumulative mass corresponding to a 5.0 micrometer diameter particle is interpolated. This cumulative mass for a single dose of powder, contained in one or more capsules, actuated into the NGI is equal to the fine particle dose less than 5.0 microns (FPD<5.0 microns).

Emitted Geometric or Volume Diameter. The volume median diameter (Dv50) of the powder after it is emitted from a dry powder inhaler, which may also be referred to as volume median geometric diameter (VMGD), was determined using a laser diffraction technique via the Spraytec diffractometer (Malvern, Inc.). Powder was filled into size 3 capsules (V-Caps, Capsugel) and placed in a capsule based dry powder inhaler (RS01 Model 7 High resistance, Plastiape, Italy), or DPI, and the DPI sealed inside a cylinder. The cylinder was connected to a positive pressure air source with steady air flow through the system measured with a mass flow meter and its duration controlled with a timer controlled solenoid valve. The exit of the dry powder inhaler was exposed to room pressure and the resulting aerosol jet passed through the laser of the diffraction particle sizer (Spraytec) in its open bench configuration before being captured by a vacuum extractor. The steady air flow rate through the system was initiated using the solenoid valve. A steady air flow rate was drawn through the DPI typically at 60 L/min for a set duration, typically of 2 seconds resulting in 2 liters of air flow. Alternatively, the air flow rate drawn through the DPI was sometimes run at 30 L/min for 3 seconds resulting in 1.5 L of air flow. 20 L/min for 3 seconds resulting in 1 L of air flow, or 15 L/min for 4 seconds resulting in 1 L of air flow. The resulting geometric particle size distribution of the aerosol was calculated from the software based on the measured scatter pattern on the photodetectors with samples typically taken at 1000 Hz for the duration of the inhalation. The Dv50, GSD, FPF<5.0 μm measured were then averaged over the duration of the inhalation.

The Emitted Dose (ED) refers to the mass of therapeutic which exits a suitable inhaler device after a firing or dispersion event. The ED is determined using a method based on USP Section 601 Aerosols, Metered-Dose Inhalers and Dry Powder Inhalers, Delivered-Dose Uniformity, Sampling the Delivered Dose from Dry Powder Inhalers, United States Pharmacopoeia convention, Rockville, Md., 13th Revision, 222-225, 2007. Contents of capsules are dispersed using the RS01 HR inhaler at a pressure drop of 4 kPa and a typical flow rate of 60 LPM and the emitted powder is collected on a filter in a filter holder sampling apparatus. The sampling apparatus is rinsed with a suitable solvent such as water and analyzed using an HPLC method. For gravimetric analysis a shorter length filter holder sampling apparatus is used to reduce deposition in the apparatus and the filter is weighed before and after to determine the mass of powder delivered from the DPI to the filter. The emitted dose of therapeutic is then calculated based on the content of therapeutic in the delivered powder. Emitted dose can be reported as the mass of therapeutic delivered from the DPI or as a percentage of the filled dose.

Capsule Emitted Powder Mass. A measure of the emission properties of the powders was determined by using the information obtained from the Andersen Cascade Impactor tests or emitted geometric diameter by Spraytec. The filled capsule weight was recorded at the beginning of the run and the final capsule weight was recorded after the completion of the run. The difference in weight represented the amount of powder emitted from the capsule (CEPM or capsule emitted powder mass). The CEPM was reported as a mass of powder or as a percent by dividing the amount of powder emitted from the capsule by the total initial particle mass in the capsule. The standard CEPM was measured for a set duration for a steady air flow rate was drawn through the DPI, typically at 60 L/min for a set duration, typically of 2 seconds resulting in 2 liters of air flow. Alternatively, the air flow rate drawn through the DPI was sometimes run at 30 L/min for 3 seconds resulting in 1.5 L of air flow, 20 L/min for 3 seconds resulting in 1 L of air flow, or 1.5 L/min for 4 seconds resulting in 1 L of air flow.

Tap Density. Tap density was measured using a modified method requiring smaller powder quantities, following USP <616> with the substitution of a 1.5 cc microcentrifuge tube (Eppendorf AG, Hamburg, Germany) or a 0.3 cc section of a disposable serological polystyrene micropipette (Grenier Bio-One, Monroe, N.C.) with polyethylene caps (Kimble Chase, Vineland, N.J.) to cap both ends and hold the powder. Instruments for measuring tap density, known to those skilled in the art, include but are not limited to the Dual Platform Microprocessor Controlled Tap Density Tester (Vankel, Cary, N.C.) or a SOTAX Tap Density Tester model TD2 (Horsham, Pa.). Tap density is a standard, approximated measure of the envelope mass density. The envelope mass density of an isotropic particle is defined as the mass of the particle divided by the minimum spherical envelope volume within which it can be enclosed.

Bulk Density. Bulk density was estimated prior to tap density measurement procedure by dividing the weight of the powder by the volume of the powder, as estimated using the volumetric measuring device.

Thermogravimetric Analysis. Thermogravimetric analysis (TGA) was performed using a Thermogravimetric Analyzer Q500 (TA Instruments, New Castle, Del.). The samples were placed into an open aluminum DSC pan with the tare weight previously recorded by the instrument. The following method was employed: Ramp 10.00° C./min from ambient (˜35° C.) to 200° C. The weight loss was reported as a function of temperature up to 150° C. TGA allows for the calculation of the water content of the dry powder.

Itraconazole Content using HPLC. The high performance liquid chromatography (HPLC) method utilizing a reverse phase C18 column coupled to an ultra violet (UV) detector is used for the identification, bulk content, assay, CUPMD and impurities analysis of dry powders containing Itraconazole. The method is also used for the analysis of Itraconazole drug substance. The reverse phase column is equilibrated to 30° C. and an autosampler set to 5° C. The mobile phases, 20 mM sodium phosphate monobasic at a pH of 2.0 (mobile phase A) and acetonitrile (mobile phase B) are used in a gradient elution from a ratio of 59:41 (A:B) to 5:95 (A:B), over the course of a 19.5 minute run time. The total method run time is 24 minutes. Detection is by UV at 258 n, and the injection volume is 10 μL. Samples and standards are be prepared and kept in amber glass containers to protect from light. PUR1900 bulk powder and drug product samples are prepared by dissolution in 70:30 acetonitrile: water. Identification is confirmed by comparing the retention time of the Itraconazole peak in PUR1900 dry powder samples to that of the Itraconazole reference standard.

Liquid Feedstock Preparation for Spray Drying. Spray drying homogenous particles requires that the ingredients of interest be solubilized in solution, or suspended or emulsion in a uniform and stable suspension or emulsion. Sodium sulfate, sodium chloride, leucine, and mannitol are sufficiently water-soluble to prepare suitable spray drying solutions. Itraconazole has very poor water-solubility. As such, a tetrahydrofuran/water co-solvent system was identified to dissolve sodium sulfate, sodium chloride, leucine, mannitol and itraconazole. Tetrahydrofuran was chosen as the co-solvent, although another organic solvent may also be suitable. When combined, the water phase and the THF phase form either a solution or a stable suspension or emulsion, depending on the relative amounts of the components in each phase.

Spray Drying Using Büchi Spray Dryer. Dry powders were prepared by spray drying on a Büchi B-290 Mini Spray Dryer (BÜCHI Labortechnik AG, Flawil, Switzerland) with powder collection from either a standard or High Performance cyclone. The system was run either with air or nitrogen as the drying and atomization gas in open-loop (single pass) mode. When run using air, the system used the Büchi B-296 dehumidifier to ensure stable temperature and humidity of the air used to spray dry. When run using nitrogen, a pressurized source of nitrogen was used. Furthermore, the aspirator of the system was adjusted to maintain the system pressure at −2.0″ water column. Atomization of the liquid feed utilized a Büchi two-fluid nozzle with a 1.5 mm diameter or a Schlick 970-0 atomizer with a 0.5 mm liquid insert (Düsen-Schlick GmbH, Coburg, Germany). Inlet temperature of the drying gas can range from 100° C. to 220° C. and outlet temperature from 30° C. to 120° C. with a liquid feedstock flowrate of 3 mL/min to 10 mL/min. The two-fluid atomizing gas ranges from 25 mm to 45 mm (300 LPH to 530 LPH) for the Büchi two-fluid nozzle and for the Schlick atomizer an atomizing air pressure of upwards of 0.3 bar. The aspirator rate ranges from 50% to 100%.

Spray Drying Using Niro Spray Dryer. Dry powders were produced by spray drying utilizing a Niro Mobile Minor spray dryer (GEA Process Engineering Inc., Columbia, Md.) with powder collection from a cyclone, a product Filter or both. Atomization of the liquid feed was performed using a co-current two-fluid nozzle either from Niro (GEA Process Engineering Inc., Columbia, Md.), although other two-fluid nozzle setups are also possible. In some embodiments, the two-fluid nozzle can be in an internal mixing setup or an external mixing setup. Additional atomization techniques include rotary atomization or a pressure nozzle. The liquid feed was fed using gear pumps (Cole-Partner Instrument Company, Vernon Hills, Ill.) directly into the two-fluid nozzle or into a static mixer (Charles Ross & Son Company, Hauppauge, N.Y.) immediately before introduction into the two-fluid nozzle. An additional liquid feed technique includes feeding from a pressurized vessel. Nitrogen or air may be used as the drying gas, provided that moisture in the air is at least partially removed before its use. Pressurized nitrogen or air can be used as the atomization gas feed to the two-fluid nozzle. The drying gas inlet temperature can range from 70° C. to 300° C. and outlet temperature from 30° C. to 120° C. with a liquid feedstock rate of 10 mL/min to 100 mL/min. The gas supplying the two-fluid atomizer can vary depending on nozzle selection and for the Niro co-current two-fluid nozzle can range from 5 kg/hr to 50 kg/hr or for the Spraying Systems two-fluid nozzle with gas cap 67147 and fluid cap 2850SS can range from 30 g/min to 150 g/min. The atomization gas rate can be set to achieve a certain gas to liquid mass ratio, which directly affects the droplet size created. The pressure inside the drying drum can range from +3″ WC to −6″ WC. Spray dried powders can be collected in a container at the outlet of the cyclone, onto a cartridge or baghouse filter, or from both a cyclone and a cartridge or baghouse filter.

Process gas as used in these descriptions refers to the sum of the drying gas and atomization gas.

### EXAMPLE 1

### Itraconazole and Salt-Containing Dry Powder Formulations

### A. Powder Preparation.

Feedstock mixtures were prepared and used to manufacture dry powders comprised of neat, dry particles containing itraconazole, leucine and either sodium sulfate (Formulation I) or sodium chloride (Formulation 11). Table 2 lists the components of the feedstock formulations used in preparation of the dry powders containing particles. Weight percentages are given on a dry basis.

TABLE 2
Feedstock compositions

| Formulation | Feedstock Composition (w/w), dry basis |
|---|---|
| I | 50.0% itraconazole (ITZ), 35.0% sodium sulfate, |
| | 15.0% leucine |
| II | 50.0% ITZ, 40.0% sodium chloride, 10.0% leucine |

The feedstock solutions that were used to spray dry particles were made as follows. For Formulation I, the liquid feedstock was batch mixed by dissolving itraconazole into tetrahydrofuran (THF) and sodium sulfate and leucine into water. The THF phase and the water phase were then mixed together. The resulting mixture had a concentration 10 g/L and a total volume of 0.80 L. This mixture was then used as a feedstock for spray drying. For Formulation II, the liquid feedstock was batch mixed by dissolving itraconazole into tetrahydrofuran (THF) and sodium chloride and leucine into water. The THF phase and the water phase were then mixed together. The resulting mixture had a concentration 10 g/L and a total volume of 4.0 L. This mixture was then used as a feedstock for spray drying.

Dry powders of Formulations I and II were manufactured from these feedstocks by spray drying on the Büchi B-290 Mini Spray Dryer (BÜCHI Labortechnik AG, Flawil, Switzerland) with cyclone powder collection. The system was run in open-loop (single pass) mode using nitrogen as the drying and atomization gas. Atomization of the liquid feed utilized a Schlick 970-0 atomizer with a 0.5 mm liquid insert. The aspirator of the system was adjusted to maintain the system pressure at −2.0″ water column.

The following spray drying conditions were followed to manufacture the dry powders. For Formulation I, the liquid feedstock solids concentration was 10 g/L, the drying gas inlet temperature was 87° C. to 94° C., the process gas outlet temperature was 40° C., the drying gas flowrate was 17.0 kg/hr, the atomization gas flowrate was 1.698 kg/hr, and the liquid feedstock flowrate was 10 mL/min. The resulting dry powder formulations are reported in Table 3. For Formulation H, the liquid feedstock solids concentration was 10 g/L, the drying gas inlet temperature was 90° C. to 94° C., the process gas outlet temperature was 40° C., the drying gas flowrate was 17.0 kg/hr, the atomization gas flowrate was 1.698 kg/hr, and the liquid feedstock flowrate was 10.0 mL/min. The resulting dry powder formulations are reported in Table 3.

TABLE 3
Dry Powder compositions, dry basis

| Formulation | Dry Powder Composition (w/w), dry basis |
|---|---|
| I | 50.0% itraconazole (ITZ), 35.0% sodium sulfate, |
| | 15.0% leucine |
| II | 50.0% ITZ, 40.0% sodium chloride, 10.0% leucine |

B. Powder Characterization.

Formulation I and II dry powders were produced by spray drying on the Büchi B-290 Mini Spray Dryer (BÜCHI Labortechnik AG, Flawil, Switzerland) with powder collection from a High Performance cyclone in a 60 mL glass vessel. Atomization of the liquid feed utilized a Büchi two-fluid nozzle with a 1.5 mm diameter. The two-fluid atomizing gas was set at 40 mm (1.698 kg/hr). Nitrogen was used as the drying gas and the atomization gas. Table 4 below includes details about the spray drying conditions.

TABLE 4
Spray Drying Process Conditions

Formulations

| Process Parameters | I | II |
|---|---|---|
| Liquid feedstock solids concentration (g/L) | 10 | 10 |
| Drying gas inlet temperature (° C.) | 87-94 | 90-94 |
| Process gas outlet temperature (° C.) | 40-41 | 40-42 |
| Drying gas flowrate (kg/hr) | 17.0 | 17.0 |
| Atomization gas flowrate (kg/hr) | 1.698 | 1.698 |
| Liquid feedstock flowrate (mL/min) | 10 | 10 |

Powder physical and aerosol properties are summarized in Tables 5 to 9 below. Values with ± indicate standard deviation of the value reported.

Formulations I and II had a tapped density greater than 0.40 g/cc. Formulations I and II had a Hausner Ratio greater than 1.6, (see Table 5).

TABLE 5
Density properties

| | Density | | |
|---|---|---|---|
| | Bulk | Tapped | Hausner |
| Formulation | g/cc | g/cc | Ratio |
| I | 0.25 ± 0 | 0.42 ± 0 | 1.68 |
| II | 0.25 ± 0.02 | 0.44 ± 0.06 | 1.76 |

Table 6 shows that Formulations I and II had a Dv50 of less than 2.5 microns at 60 LPM ran for 2 seconds. Formulations I and II had a Dv50 of less than 3.0 μm at 30 LPM ran for 3 seconds.

TABLE 6
Geometric Diameters

| | Dispersibility - Spraytec | | | |
|---|---|---|---|---|
| | @ 60 LPM | | @ 30 LPM | |
| Formulation | Dv50 (μm) | GSD | Dv50 (μm) | GSD |
| I | 1.80 ± 0.01 | 2.43 ± 0.13 | 2.77 ± 0.03 | 2.86 ± 0.06 |
| II | 2.04 ± 0.05 | 2.63 ± 0.25 | 2.98 ± 0.07 | 3.28 ± 0.28 |

Table 7 shows that Formulations I and II had a capsule emitted particle mass (CEPM) of greater than 98% at 60 LPM. Formulations I and II had a CEPM of greater than 94% at 30 LPM.

TABLE 7
Dispersibility properties

Dispersibility - CEPM

| | @ 60 LPM | @ 30 LPM |
|---|---|---|
| Formulation | CEPM | CEPM |
| I | 100.2% ± 1.0% | 96.5% ± 0.7% |
| II | 98.2% ± 0.3% | 94.1% ± 1.4% |

Table 8 shows that Formulations I and II had a Dv50 of less than 2.0 μm when using the RODOS at a 1.0 bar setting. Formulations I and II had a RODOS Ratio for 0.5 bar/4 bar of less than 1.2. Formulations I and II had a RODOS Ratio for 1 bar/4 bar of less than about 1.1.

TABLE 8
Dispersibility properties (Geometric diameter using RODOS)

| | RODOS | | | | | | | |
|---|---|---|---|---|---|---|---|---|
| | 0.5 bar | | 1.0 bar | | 4.0 bar | | | |
| | Dv50 | | Dv50 | | Dv50 | | | |
| Form. | (μm) | GSD | (μm) | GSD | (μm) | GSD | 0.5/4 bar | 1/4 bar |
| I | 1.86 | 2.12 | 1.68 | 2.05 | 1.61 | 2.01 | 1.16 | 1.05 |
| II | 1.98 | 2.33 | 1.79 | 2.29 | 1.67 | 2.21 | 1.18 | 1.07 |

Table 9 shows that Formulations I and II had a Dv50 of less than 2.0 μm when using the RODOS at a 1.0 bar setting. Formulations I and II had a RODOS Ratio for 0.5 bar/4 bar of less than 1.4. Formulations 1 and II had a RODOS Ratio for 1 bar/4 bar of less than or equal to about 1.1.

TABLE 9
Formulations I and II Aerodynamic Size Characteristics

| | | | Formulation I | Formulation II |
|---|---|---|---|---|
| | | | n = 1 Lot | n = 1 Lot |
| Test Method | Parameter | Unit | mean ± SD | mean ± SD |
| Aerodynamic | Capsules | (μg | 76.25 ± 55.51 | 259.75 ± 5.30 |
| Particle Size | | ITZ) | | |
| using an | −1 | (μg | 104.25 ± 15.91 | 133.25 ± 40.66 |
| ACI-8 | | ITZ) | | |
| | 0 | (μg | 254.75 ± 62.58 | 858.00 ± 725.49 |
| | | ITZ) | | |
| | 1 | (μg | 862.75 ± 98.64 | 630.50 ± 743.17 |
| | | ITZ) | | |
| | 2 | (μg | 1079.75 ± 32.88 | 1236.50 ± 33.23 |
| | | ITZ) | | |
| | 3 | (μg | 2040.25 ± 190.57 | 1834.50 ± 0.71 |
| | | ITZ) | | |
| | 4 | (μg | 1335.50 ± 43.84 | 1071.00 ± 131.52 |
| | | ITZ) | | |
| | 5 | (μg | 538.25 ± 49.85 | 371.50 ± 13.44 |
| | | ITZ) | | |
| | 6 | (μg | 33.50 ± 4.95 | 24.75 ± 6.01 |
| | | ITZ) | | |
| | F | (μg | 2.25 ± 3.18 | 0.00 ± 0.00 |
| | | ITZ) | | |
| | MMAD | (μm) | 2.78 ± 0.09 | 3.14 ± 0.06 |
| | GSD | — | 1.89 ± 0.06 | 2.55 ± 0.96 |
| | FPD < 2.0 μm | (μg | 1909.50 ± 101.82 | 1467.25 ± 138.95 |
| | | ITZ) | | |
| | FPF_TD < 2.0 μm | (%) | 19.1 ± 1.0 | 14.7 ± 1.4 |
| | FPD < 5.0 μm | (μg | 5276.0 ± 297.09 | 4718.39 ± 39.45 |
| | | ITZ) | | |
| | FPF_TD < 5.0 μm | (%) | 52.8 ± 3.0 | 47.2 ± 0.4 |
| | FPD < 2.0 μm/ | — | 0.36 ± 0.00 | 0.31 ± 0.03 |
| | FPD < 5.0 μm | | | |

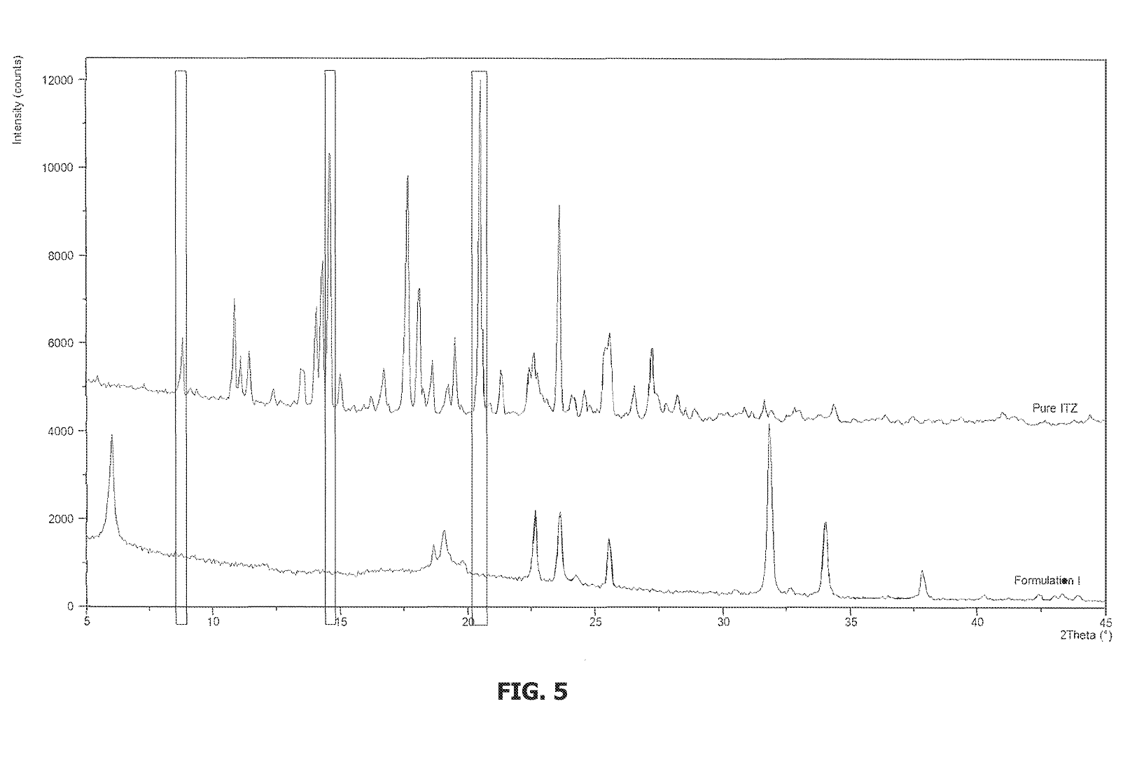

The crystallinity of Formulation 1 was assessed via XRD (FIG. 5). No itraconazole peaks are observed, indicating no appreciable levels of itraconazole are present in the formulation. As shown, all peaks observed in the formulation correspond to the excipients. The solid state of the itraconazole in Formulation 1 can therefore be characterized as amorphous.

### EXAMPLE 2

### Pulmonary Delivery of Formulation I in a 7-Day Inhalation Study in Rats Enables High Lung Exposure of Itraconazole Relative to Oral Dosing

Pulmonary aspergillosis is a common infection that can result in significant morbidity and mortality in patients with asthma, especially severe asthma, and cystic fibrosis. Depending on the state of the host, infection can result in Aspergillus bronchitis or Allergic Bronchopulmonary Aspergillosis (ABPA), each of which are associated with diminished lung function and pulmonary exacerbations. Standard of care generally calls for oral doses of triazoles that inhibit fungal cytochrome p450s and inhibit fungal growth. Itraconazole (ITZ) and other triazoles are commonly used as first line therapies for fungal infections. These treatments are generally poorly and variably bioavailable and result in a number of systemic side effects that limit their therapeutic potential. In addition, ITZ and other triazoles have an extensive list of drug-drug interactions that require drug monitoring during treatment and may lead to hepatotoxicity. We hypothesized that inhaled delivery of ITZ as a dry powder (Formulation I) would result in significantly higher lung exposure and lower systemic exposure relative to oral dosing. This profile may allow for improved efficacy, reduced overall dose and reduced drug-drug interactions.

A 7-day repeat dose study in rats was performed to assess lung and systemic exposure levels of ITZ following inhaled (Formulation I) or oral (Sporanox) exposure. Oral ITZ (Sporanox® solution, Janssen Pharmaceuticals, 10 mg/mL) was dosed daily via oral gavage at 5 mg/kg/day. PUR1900, a novel dry powder formulation of ITZ was delivered by nose-only inhalation with an average achieved delivered dose of 5.2 mg/kg/day. In rodent dosing, approximately 10% of the delivered dose is considered to have reached the lung, with the remaining 90% filtered by the nasopharynx and ultimately likely swallowed. Blood samples were collected on Days 1 and 7 at pre-dose, immediately after dose and 2, 4, 8 and 24 hours after dose. Lung tissue was collected on Day 1 immediately after dose and 24 hours after dose and on Day 7, 24 hours after dose for measurement of itraconazole and the active metabolite, hydroxy-itraconazole (OH-ITZ).

Three groups of male and female rats received Formulation I by daily inhalation administration over a 1-hour exposure period at target dose levels of 5, 20 or 44 mg/kg/day (of active ingredient, itraconazole) for 7 days. A further group of rats received an oral formulation (Sporanox) via oral gavage at a target dose level of 5 mg/kg/day for 7 days in order to compare the systemic and lung exposure of the test article via the inhaled and oral routes. Blood and lung samples were taken on Day 1 and Day 7 to assess the pulmonary and systemic exposure of male and female rats to itraconazole and hydroxy-itraconazole.

The rate (Cₘₐₓ) and extent (AUC_{0-25 h}) of systemic exposure of rats to itraconazole and its metabolite hydroxy-itraconazole were characterized by nonlinear (dose-dependent) kinetics over the target dose range 5 to 44 mg/kg/day on Day 1 and Day 7 of the 7-day inhalation toxicity study. Increasing the dose of Formulation 1 above 5 mg/kg/day resulted in a lower systemic exposure than would be predicted from a linear relationship. Toxicokinetic summaries of the systemic exposure to itraconazole and hydroxy itraconazole are shown in the tables below.

TABLE 10
Maximum plasma itraconazole concentration (Cₘₐₓ) and 24-hour exposure (AUC_{0-25 h})
in male and female rats after inhaled Formulation I (Form I) or oral Sporanox (Spor).

| | Target | | | | | | | | |
|---|---|---|---|---|---|---|---|---|---|
| | delivered | Cₘₐₓ (ng/mL) | | | | AUC_{0-25 h} (ng · h/mL) | | | |
| | dose level | Day 1 | | Day 7 | | Day 1 | | Day 7 | |
| Dose route | (mg/kg/day) | Males | Females | Males | Females | Males | Females | Males | Females |
| Inhalation | 5 (Form I) | 120 | 257 | 78.0 | 436 | 995 | 2880 | 721 | 7580 |
| Inhalation | 20 (Form I) | _^{a} | _^{a} | 356 | 1330 | _^{a} | _^{a} | 2600 | 21200 |
| Inhalation | 44 (Form I) | 629 | 916 | 490 | 3720 | 5470 | 17700 | 5980 | 58200 |
| Oral | 5 (Spor) | 114 | 296 | 138 | 737 | 1580^{b} | 4590^{b} | 2020^{b} | 14000^{b} |

^{a}Due to technical issues, no samples were taken from these animals until 8 hours post inhalation on Day 1, therefore Cₘₐₓ and AUC₀₋₂₅ values were not calculated.
^{b}AUC_{0-24 h} values.

TABLE 11
Maximum plasma hydroxy-itraconazole concentration (Cₘₐₓ) and 24-hour
exposure (AUC_{0-25 h}) in male and female rats after inhaled Formulation I or oral Sporanox.

| | Target | | | | | | | | |
|---|---|---|---|---|---|---|---|---|---|
| | delivered | Cₘₐₓ (ng/mL) | | | | AUC_{0-25 h} (ng · h/mL) | | | |
| | dose level | Day 1 | | Day 7 | | Day 1 | | Day 7 | |
| Dose route | (mg/kg/day) | Males | Females | Males | Females | Males | Females | Males | Females |
| Inhalation | 5 (Form I) | 251 | 359 | 231 | 698 | 3670 | 5040 | 3210 | 13000 |
| Inhalation | 20 (Form I) | _^{a} | _^{a} | 516 | 1320 | _^{a} | _^{a} | 7770 | 25000 |
| Inhalation | 44 (Form I) | 717 | 1130 | 745 | 2710 | 11600 | 23300 | 14300 | 51700 |
| Oral | 5 (Spor) | 458 | 680 | 481 | 891 | 6500^{b} | 10000^{b} | 7250^{b} | 17300^{b} |

^{a}Due to technical issues, no samples were taken from these animals until 8 hours post inhalation on Day 1, therefore Cₘₐₓ and AUC_{0-25 h} values were not calculated.
^{b}AUC_{0-24 h} values

The study provided evidence that, following both oral and inhaled dosing, the systemic exposure of female rats to itraconazole and hydroxy-itraconazole was higher than that of males and that there was no systemic accumulation in males, but there was systemic accumulation of itraconazole and hydroxy-itraconazole after repeated oral or inhaled administration in females.

Systemic exposure of rats to itraconazole and hydroxy-itraconazole was lower following inhalation exposure at the target 5 mg/kg/day compared to following oral gavage administration at the same dose level.

For Formulation I at the various inhaled doses, the concentrations of itraconazole in the lung were much greater than in the blood immediately after dose and declined over the 24 hours. Over the course of 7 days of both oral and inhaled dosing, lung accumulation was evident at all doses as shown by the higher levels 24 hours after dose on Day 7 relative to 24 hours after dose on Day 1. Hydroxy-itraconazole levels in the lung were much lower than itraconazole levels immediately after inhaled dose and increased over the course of the dosing period, also showing lung accumulation on Day 7 relative to Day 1. Lung accumulation after oral gavage dose was only evident for female rats. Lung exposure for itraconazole and hydroxy-itraconazole are summarized below.

TABLE 12
Lung itraconazole concentrations immediately after dose on Day 1 and 24 hours
after dose on Days 1 and 7 in male and female rats after inhaled Formulation I or oral
Sporanox.

| | Target delivered | Lung tissue concentration (ng/g) | | | | | |
|---|---|---|---|---|---|---|---|
| | dose level | Day 1 IAD | | Day 1 24 hr | | Day 7 24 hour | |
| Dose route | (mg/kg/day) | Males | Females | Males | Females | Males | Females |
| Inhalation | 5 (Form I) | 13000 | 6300 | 32 | 385 | 156 | 719 |
| Inhalation | 20 (Form I) | _^{a} | _^{a} | 150 | 347 | 877 | 1960 |
| Inhalation | 44 (Form I) | 54700 | 52500 | 693 | 2720 | 2490 | 6110 |
| Oral | 5 (Spor) | 64.1 | 0 | 24.3 | 432 | 19.1 | 1240 |

^{a}Due to technical issues, no samples were taken from these animals until 8 hlours post inhalation on Day 1.

TABLE 13
Lung hydroxy-itraconazole concentrations immediately after dose on Day 1 and
24 hours after dose on Days 1 and 7 in male and female rats after inhaled Formulation I
or oral Sporanox.

| | Target delivered | Lung tissue concentration (ng/g) | | | | | |
|---|---|---|---|---|---|---|---|
| | dose level | Day 1 IAD | | Day 1 24 hr | | Day 7 24 hour | |
| Dose route | (mg/kg/day) | Males | Females | Males | Females | Males | Females |
| Inhalation | 5 (Form I) | 34.7 | 194 | 138 | 480 | 196 | 634 |
| Inhalation | 20 (Form I) | _^{a} | _^{a} | 364 | 442 | 142 | 706 |
| Inhalation | 44 (Form I) | 450 | 632 | 428 | 1460 | 960 | 2510 |
| Oral | 5 (Spor) | 40.7 | 0 | 130 | 455 | 151 | 1100 |

^{a}Due to technical issues, no samples were taken from these animals until 8 hours post inhalation on Day 1.

Like with systemic exposure, lung exposure of female rats to itraconazole and hydroxy-itraconazole was generally higher than that of males after both inhaled and oral dosing, though lung accumulation in males and females was similar in most cases.

Lung exposure of rats to itraconazole hut not hydroxy-itraconazole immediately after dose was higher following inhalation exposure at the target 5 mg/kg/day compared to following oral gavage administration at the same dose level.

### EXAMPLE 3

### 7-Day Inhalation Study in Dogs

Male and female dogs, 1/sex at each dose level, received Formulation I by daily inhalation administration over a 1-hour exposure period at target dose levels of 5, 10 or 20 mg/kg/day (of active ingredient, itraconazole) for 7 days. A further pair of dogs received an oral formulation (Sporanox®, Janssen Pharmaceuticals) via oral gavage at a target dose level of 5 mg/kg/day for 7 days in order to compare the systemic and lung exposure of the test article via the inhaled and oral routes. Blood samples were taken on Day 1 and Day 7 in order to assess the systemic exposure to itraconazole and its metabolite hydroxy-itraconazole. Lung samples were also collected on Day 7 only, 24 hours after the last dose in order to compare lung exposure between dose routes.

The rate (Cₘₐₓ) and extent (AUC_{0-25 h}) of systemic exposure of dogs to itraconazole or hydroxy-itraconazole via the inhaled route generally increased in a less than dose proportional manner on Day 1. On Day 7, however, the Cₘₐₓ and AUC_{0-25 h} increased with increasing dose and these increases were generally dose proportionate or greater than dose proportionate. There were no sex differences in systemic exposure in dogs. Toxicokinetic summaries of the systemic exposure to itraconazole and hydroxy itraconazole are shown in the tables below.

TABLE 14
Maximum plasma itraconazole concentration (Cₘₐₓ) and 24-hour exposure (AUC_{0-25 h})
in male and female dogs after inhaled Formulation I (Form I) or oral Sporanox (Spor).

| | Target | Cₘₐₓ (ng/mL)* | | | | AUC_{0-25 h} (ng · h/mL)* | | | |
|---|---|---|---|---|---|---|---|---|---|
| | dose level | Day 1 | | Day 7 | | Day 1 | | Day 7 | |
| Dose route | (mg/kg/day) | Males | Females | Males | Females | Males | Females | Males | Females |
| Inhalation | 5 (Form I) | 254 | 233 | 362 | 343 | 2840 | 3300 | 4870 | 5430 |
| Inhalation | 10 (Form I) | 202 | 195 | 408 | 705 | 3410 | 3370 | 5550 | 8220 |
| Inhalation | 20 (Form I) | 283 | 303 | 1170 | 1730 | 4520 | 3200 | 25500 | 34500 |
| Oral | 5 (Spor) | 458 | 485 | 1500 | 810 | 6400 | 6220 | 17500 | 12200 |

*Mean values presented for Group 1 (5 mg/kg/day; inhalation)

TABLE 15
Maximum plasma hydroxy-itraconazole concentration (Cₘₐₓ) and 24-hour
exposure (AUC_{0-25 h}) in male and female dogs after inhaled Formulation I or oral Sporanox.

| | Target | Cₘₐₓ (ng/mL)* | | | | AUC_{0-25 h} (ng · h/mL)* | | | |
|---|---|---|---|---|---|---|---|---|---|
| | dose level | Day 1 | | 7 | | Day 1 | | 7 | |
| Dose route | (mg/kg/day) | Males | Females | Males | Females | Males | Females | Males | Females |
| Inhalation | 5 (Form I) | 106 | 117 | 312 | 325 | 1890 | 2270 | 5910 | 6470 |
| Inhalation | 10 (Form I) | 122 | 95.8 | 325 | 520 | 2030 | 1960 | 6620 | 9710 |
| Inhalation | 20 (Form I) | 148 | 96.7 | 1240 | 1630 | 2710 | 1870 | 26600 | 33800 |
| Oral | 5 (Spor) | 198 | 207 | 695 | 689 | 3590 | 4220 | 13600 | 14400 |

*Mean values presented for Group 1 (5 mg/kg/day; inhalation)

For Formulation I at the various inhaled doses, when compared to similar deposited doses in the rat, the concentrations of itraconazole and hydroxy-itraconazole in the lung were similar to those found in the rat, suggesting a similar accumulation propensity. Hydroxy-itraconazole levels in the lung were much lower than itraconazole levels demonstrating that most or all of the hydroxy-itraconazole in the lung comes from the systemic circulation with little to no metabolism in the lung. Lung exposure for itraconazole and hydroxy-itraconazole are summarized in Table 16.

TABLE 16
Lung itraconazole concentrations 24 hours after dose on Day 7 in male
and female dogs after inhaled Formulation I or oral Sporanox.

| | Target | | Itraconazole | | Hydroxy Itraconazole | |
|---|---|---|---|---|---|---|
| | dose level | | Day 7 (ng/g) | | Day 7 (ng/g) | |
| Dose route | (mg/kg/day) | | Males | Females | Males | Females |
| Inhalation | 5 | (Form I) | 6390 | 4110 | 758 | 365 |
| Inhalation | 10 | (Form I) | 3850 | 2440 | 653 | 580 |
| Inhalation | 20 | (Form I) | 17700 | 8310 | 2920 | 3000 |
| Oral | 5 | (Spor) | 695 | 908 | 725 | 1130 |

Individual values are presented from 1 dog/sex/group.

### EXAMPLE 4

### Efficacy of inhaled Formulation I in a Model of Invasive Pulmonary Aspergillosis

Immunosuppressed guinea-pigs were exposed to a 45-minute aerosol challenge with Aspergillus conidia, resulting in an active pulmonary aspergillosis. Starting 1 day after the Aspergillus challenge, groups of guinea-pigs received a placebo (positive control) or Formulation 1 by daily inhalation administration over a 1-hour exposure period at target dose levels of 10 or 30 mg/kg/day (of active ingredient, itraconazole) for 10 days. A further group of guinea pigs received an oral formulation (Sporanox®, Janssen Pharmaceuticals) via oral gavage at a target dose level of 10 mg/kg/day for 10 days. Clinical signs, and survival were monitored throughout the 10 days of dosing and for up to 4 days after the end of dosing.

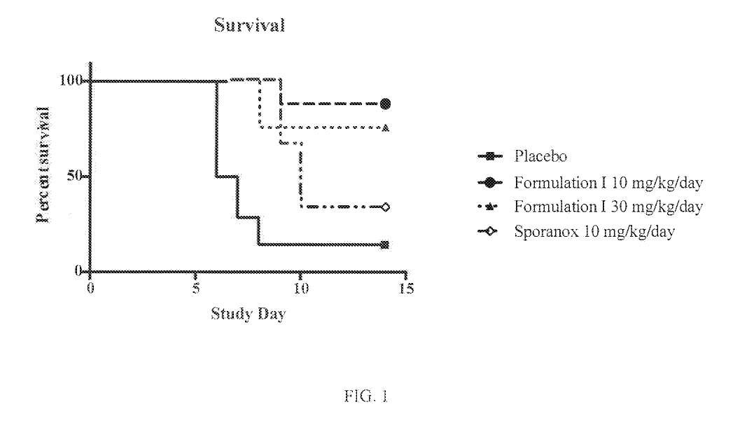

The onset of clinical signs in groups receiving both inhaled and oral itraconazole was delayed versus the placebo group and approximately 33% of itraconazole treated animals exhibited no clinical signs. Clinical signs in moribund animals were consistent with disease, namely, decreased grooming behavior, lethargy, respiratory distress, increasing weight loss, and increasing temperature as the study progressed. Survival was significantly increased in all Formulation I groups compared to the placebo and Sporanox-treated animals. Among placebo control animals. 6 of 8 died between days 6 and 8, with an average day of death of 9 days. Two control animals survived to termination of the study. Four of 6 Sporanox treated animals died between days 9 and 10 with an overall average day of death of day 11. Of Formulation I-treated animals, death occurred in only 1 of 8 at 10 mg/kg/day, 2 of 8 at 30 mg/kg/day. The average day of death in these animals was day 13+ days with the majority surviving until scheduled termination. These data (FIG. 1) indicate the potential of Formulation I as an inhaled therapy for pulmonary Aspergillus infection.

### EXAMPLE 5

### Single Dose Inhalation PK Study in Rats

### A. Materials and Methods

Blood and lung tissue samples were taken from rats following a single inhalation administration of each of five different itraconazole formulations over a 60-minute exposure period in order to assess the systemic exposure of male rats to itraconazole and its metabolite, hydroxy-itraconazole, at a nominal dose level of 5 mg/kg. Plasma concentrations of itraconazole and hydroxy-itraconazole in samples taken at the end of the exposure period, and up to 96 hours after the end of exposure were measured by validated LC-MS/MS methods.

### B. Results—Plasma

Maximum mean plasma concentrations (Cₘₐₓ) of itraconazole and the areas under the mean plasma concentration-time curves estimated up to the time of the last quantifiable sample (AUCₗₐₛₜ) are summarized in Table 17.

TABLE 17
Plasma Cₘₐₓ and AUCₗₐₛₜ

Itraconazole

| Formulation | Cₘₐₓ (ng/mL) | AUCₗₐₛₜ (ng · h/mL) |
|---|---|---|
| I | 123 | 1170 |
| XV | 7.65 | 157 |

The ratios of the maximum mean plasma concentrations (Cₘₐₓ) and areas under the mean plasma concentration-time curves (AUCₗₐₛₜ) in each group relative to the Cₘₐₓ and AUCₗₐₛₜ values for the group receiving Formulation 1, based on Cₘₐₓ and AUCₗₐₛₜ values corrected for the differences in the doses received, are presented in Table 18.

TABLE 18
Plasma Cₘₐₓ and AUCₗₐₛₜ, both relative to Formulation I.

Itraconazole

| Formulation | Cₘₐₓ (ng/mL) | AUCₗₐₛₜ (ng · h/mL) |
|---|---|---|
| I | 1 | 1 |
| XV | 0.056 | 0.12 |

The rate (Cₘₐₓ) and extent (AUCₗₐₛₜ) of systemic exposure of rats to itraconazole were highest following exposure to Formulation I. Cₘₐₓ and AUCₗₐₛₜ were lower following exposure to Formulation XV.

### CONCLUSIONS

The systemic exposure of rats to itraconzole was highest following administration of Formulation I. Systemic exposure was lower following administration of Formulation XV.

### EXAMPLE 6

### Dry Powder Formulation of Amorphous Itraconazole Prepared for Use in 28-Day Inhalation Studies

### A. Powder Preparation.

A feedstock solution utilizing a water-tetrahydrofuran (THF) co-solvent system was prepared and used to manufacture a dry powder composed of itraconazole, sodium sulfate and leucine. A drug load of 50 wt % itraconazole, on a dry basis, was targeted. The feedstock solution that was used to spray dry particles was made as follows. The required quantity of water was weighed into a suitably sized glass vessel. The excipients were added to the water and the solution allowed to stir until visually clear. The required amount of THF was weighed into a suitably sized glass vessel. The itraconazole was added to the THF and the solution allowed to stir until visually clear. The itraconazole-containing THF solution was then added to the excipient solution and stirred until visually homogenous. The feedstock was then spray-dried. The individual feedstock volume was 9.5625 L. Fourteen of these feedstocks were prepared for a total of 133.875 L which supported a manufacturing campaign of approximately 30 hours. Table 19 lists the components of each feedstock used in preparation of the thy powder.

TABLE 19
Feedstock composition

| | | | | Sodium | | |
|---|---|---|---|---|---|---|
| | Water | Tetrahydrofuran | Itraconazole | sulfate | Leucine | Total mass |
| Formulation | (g) | (g) | (g) | (g) | (g) | (gm) |
| XX | 4295.379 | 4676.636 | 57.375 | 40.163 | 17.213 | 9086.766 |

A dry powder of Formulation XX was manufactured from this feedstock by spray drying on the Niro Mobile Minor spray dryer (GEA Process Engineering Inc., Columbia, Md.) with bag filter collection. The system was run in open-loop (single pass) mode using nitrogen as the drying and atomization gas. Atomization of the liquid feed utilized a Niro atomizer with a 1.0 mm liquid insert. The aspirator of the system was adjusted to maintain the system pressure at −2.0″ water column. Formulation XX is a scaled-up version Of Formulation I, manufactured as described here in Example 6 to allow for larger production amounts.

The following spray drying conditions were followed to manufacture the dry powder. For Formulation XX, the liquid feedstock solids concentration was 12 g/L, the process gas inlet temperature was 120° C. to 140° C., the process gas outlet temperature was 40° C., the drying gas flowrate was 80 kg/hr, the atomization gas flowrate was 352.2 g/min, the atomization gas back pressure at the atomizer inlet was 45 psig to 57 psig and the liquid feedstock flowrate was 75 mL/min. The resulting dry powder formulation is reported in Table 20. The itraconazole in the formulation was amorphous.

TABLE 20
Dry powder composition, dry basis

| Formulation | Dry Powder Composition (w/w), dry basis |
|---|---|
| XX | 50% itraconazole, 35% sodium sulfate, 15% leucine |

B. Powder Characterization.

The bulk particle size characteristics for the formulation are found in Table 21. The span at 1 bar of 1.83 for Formulation XX, indicates a relatively narrow size distribution. The 1 bar/4 bar dispersibility ratio of 1.06 for Formulation XX, indicates the particle size is relatively independent of dispersion energy, a desirable characteristic which allows similar dispersion across a range of dispersion energies.

TABLE 21
Bulk particle size

| | 0.5 bar | | 1 bar | | 4 bar | | |
|---|---|---|---|---|---|---|---|
| Formu- | Dv [50] | | Dv [50] | | Dv [50] | | 1 bar:4 bar |
| lation | (μm) | Span | (μm) | Span | (μm) | Span | Dv [50] ratio |
| XX | 1.84 | 1.83 | 1.58 | 1.81 | 1.50 | 1.82 | 1.06 |

The weight loss of Formulation XX was measured via TGA and was found to be 0.34%.

The itraconazole content of Formulation XX was measured with HPLC-UV and is 100.9% of nominal.

### EXAMPLE 7

### 28-Day Inhalation Study in Rats

### A. Materials and Methods

In order to expand the understanding of the utility of Formulation XX in an animal model beyond the 7-day study in rats presented in Example 2, a 28-day study in rats was conducted where both the plasma and lung pharmacokinetics were assessed. Five groups of animals were dosed daily for 28 days with either air or placebo controls or one of three doses of Formulation XX. The groups and the achieved doses are detailed in Table 22.

TABLE 22
Dose Groups in 28-Day Study

| | | Achieved Total Delivered API Dose |
|---|---|---|
| Group No. | Formulation | Level (mg/kg) |
| 1 | Air Control | 0 |
| 2 | Placebo Control | 0 |
| 3 | XX | 5.8 |
| 4 | XX | 22 |
| 5 | XX | 49 |

In the study, blood samples were taken from rats following the first and last inhalation administration of each formulations in order to assess the lung and systemic exposure and accumulation of itraconazole in male and female rats. Plasma concentrations of itraconazole in samples were measured by validated LC-MS/MS methods.

### B. Results—Plasma

Maximum mean plasma concentrations (Cₘₐₓ) of itraconazole and the areas under the mean plasma concentration-time curves estimated up to the time of the last quantifiable sample (AUC_{0-25 h}) on Days 1 and 28 in male and female rats from the 28-day study with Formulation XX, are summarized in Table 23.

TABLE 23
Plasma Cₘₐₓ and AUC_{0-25 h} for Itraconazole

| | | Cₘₐₓ (ng/mL) | | | | AUC_{0-25 h} (ng · h/mL) | | | |
|---|---|---|---|---|---|---|---|---|---|
| | Dose level | Day 1 | | Day 28 | | Day 1 | | Day 28 | |
| Formulation | (mg/kg/day) | Males | Females | Males | Females | Males | Females | Males | Females |
| XX | 5.8 | 188 | 283 | 117 | 430 | 1100 | 3840 | 914 | 6310 |
| XX | 22.0 | 348 | 620 | 335 | 1310 | 2710 | 11200 | 2110 | 22200 |
| XX | 49.0 | 510 | 928 | 602 | 2270 | 4990 | 17800 | 3800 | 35200 |

Table 24 below summarizes the dose-normalized average Cₘₐₓ and AUC_{0-25 h} for itraconazole for each of the formulations from both 28-day studies using the target dose of 5 mg/kg/day from each study. Normalization was achieved by dividing the exposures measured by the actual achieved dose for each study on each day.

TABLE 24
Dose-normalized plasma Cₘₐₓ and AUC_{0-25 h}, for Formulation I.

| | Day 1 | | | | Day 28 | | | |
|---|---|---|---|---|---|---|---|---|
| For- | Cₘₐₓ | | AUC_{0-25 h} | | Cₘₐₓ | | AUC_{0-25 h} | |
| mu- | (ng/mL) | | (ng · h/mL) | | (ng/mL) | | (ng · h/mL) | |
| lation | M | F | M | F | M | F | M | F |
| XX | 17.40 | 28.18 | 135.50 | 509.09 | 18.61 | 68.95 | 117.22 | 1168.42 |

The Cₘₐₓ and AUC_{0-25 h} of systemic exposure increased with increasing dose in a less than dose proportionate manner on each day. The dose-adjusted Cₘₐₓ and AUC_{0-25 h} of systemic exposure of female rats to were approximately 2.7-fold and 6.3-fold higher respectively overall than those in males and tended to be more marked on Day 28.

After administration on Day 28, the Cₘₐₓ and AUC_{0-25 h} of systemic exposure of male rats to itraconazole were generally similar to those values after a single dose (Day 1). However, systemic exposure of female rats to itraconazole on Day 28 was higher than that after a single dose (Day 1). The accumulation ratios, based on AUC_{0-25 h} values corrected for the achieved doses on each sampling day were generally close to or less than one in males (0.87 to 1.5) and greater than one in females (2.1 to 2.9-fold higher), indicating that accumulation of itraconazole occurred in females after repeated inhalation exposure.

### EXAMPLE 8

### Dry Powder Formulations of Itraconazole Prepared for Use in 28-Day Inhalation Studies

A. Powder Preparation.

The microcrystalline itraconazole for Formulation XXIII was prepared using a Qualification Micronizer jet mill (Sturtevant, Hanover, Mass. USA). The feed pressure was set to 85 psig and the grind pressure was set to 45 psig. Itraconazole was continuously fed into the mill until 480.0 g of itraconazole was milled. The final median particle size (Dv(50)) of the milled API was 1640 nm. The micronized itraconazole for Formulation XXIII was then compounded into a suspension consisting of 10 wt % itraconazole and 0.25 wt % polysorbate 80 in deionized water. The batch size was 4800 g. The polysorbate 80 was dissolved in 88.75% deionized water via magnetic stir bar, then the itraconazole was slowly added and allowed to mix until the suspension was observed to be visually dispersed and homogeneous.

A feedstock suspension was prepared and used to manufacture a dry powder composed of crystalline itraconazole, and other additional excipients. A drug load of 50 wt % itraconazole, on a dry basis, was targeted. The feedstock suspension that was used to spray dry particles was made as follows. The required quantity of water was weighed into a suitably sized glass vessel. The excipients were added to the water and the solution was allowed to stir until visually clear. The itraconazole-containing suspension was then added to the excipient solution and stirred until visually homogenous. The feedstock was then spray-dried. The feedstock was stirred while spray dried. The individual feedstock mass for Formulation XXIII was 8.0 kg. The feedstock suspension was spray dried. Table 25 lists the components of the feedstock used in preparation of the dry powder.

TABLE 25
Feedstock composition for formulation containing polysorbate 80

| | | | | Sodium | | |
|---|---|---|---|---|---|---|
| | Water | Itraconazole | Polysorbate 80 | sulfate | Leucine | Total mass |
| Formulation | (g) | (g) | (g) | (g) | (g) | (gm) |
| XXIII | 7904.0 | 48.0 | 1.2 | 33.6 | 13.2 | 8000.0 |

Dry powder of Formulation XXIII was manufactured from the feedstock by spray drying on the Niro Mobile Minor spray dryer (GEA Process Engineering Inc., Columbia, Md.) with bag filter collection. The system was run in open-loop (single pass) mode using nitrogen as the drying and atomization gas. Atomization of the liquid feed utilized a Niro two fluid nozzle atomizer with a 1.0 mm liquid insert. The aspirator of the system was adjusted to maintain the system pressure at −2.0″ water column.

The following spray drying conditions were followed to manufacture the dry powder. For Formulation XXIII, the liquid feedstock solids concentration was 1.2%, the process gas inlet temperature was 170-190° C., the process gas outlet temperature was 65° C., the drying gas flowrate was 80.0 kg/hr, the atomization gas flowrate was 250.0 g/min, and the liquid feedstock flowrate was 50.0 g/min. The resulting dry powder formulation is reported in Table 26.

TABLE 26
Dry powder composition, dry basis

| | | Dry Powder Composition (w/w), |
|---|---|---|
| Formulation | Description | dry basis |
| XXIII | Microcrystalline, PS80 | 50% itraconazole, 35% sodium |
| | stabilizer | sulfate, 13.75% leucine, 1.25% |
| | | polysorbate 80 |

B. Powder Characterization.

The bulk particle size characteristics for the formulation is found in Table 27. The span at 1 bar of less than 2.05 for Formulation XXIII indicates a relatively narrow size distribution. The 1 bar/4 bar dispersibility ratio less than 1.25 for Formulation XXIII indicates that it is relatively independent of dispersion energy, a desirable characteristic which allows similar particle dispersion across a range of dispersion energies.

TABLE 27
Bulk particle size

| | 0.5 bar | | 1 bar | | 4 bar | | |
|---|---|---|---|---|---|---|---|
| Formu- | Dv [50] | | Dv [50] | | Dv [50] | | 1 bar:4 bar |
| lation | (μm) | Span | (μm) | Span | (μm) | Span | Dv [50] ratio |
| XXIII | 2.15 | 1.82 | 2.03 | 1.88 | 1.89 | 1.88 | 1.08 |

The weight loss of Formulation XXIII was measured via TGA and is detailed in Table 28.

TABLE 28
Weight loss (%) via TGA

| Formulation | Weight loss via TGA (%) |
|---|---|
| XXIII | 0.37 |

The itraconazole content of Formulation XXIII was measured with HPLC-UV and is detailed in Table 29.

TABLE 29
Itraconazole content

| Formulation | Itraconazole content (% label claim) |
|---|---|
| XXIII | 101.20 |

### EXAMPLE 9

### 28-Day Inhalation Toxicity Study in Rats

### A. Materials and Methods

In order to assess both the plasma and lung pharmacokinetics as well as the potential for local tissue toxicity, a 28-day study was performed. In the study 7 groups of rats were dosed with microcrystalline nanoparticulate itraconazole, daily for 28 days, or in the case of one group, every three days. Groups and achieved doses are detailed in Table 30.

TABLE 30
Dose Groups in 28-Day Study B

| | | Achieved Total Delivered API Dose |
|---|---|---|
| Group No. | Formulation | Level (mg/kg) |
| 5 | XXIII | 5.0 |
| 6 | XXIII | 14.5 |

Blood samples were taken from rats following the first and last inhalation administration of each formulation in order to assess the lung and systemic exposure and accumulation of itraconazole in male and female rats. In addition, pulmonary tissue samples, including the larynx, trachea, tracheal bifurcation (carina) and lungs, were collected from all animals 24 hours after the last dose in order to assess microscopic pathology changes resulting from the dosing. Plasma concentrations of itraconazole in samples were measured by validated LC-MS/MS methods.

### B. Results—Plasma

Maximum mean plasma concentrations (Cₘₐₓ) of itraconazole and the areas under the mean plasma concentration-time curves estimated up to the time of the last quantifiable sample (AUC₀₋ₗₐₛₜ) on Days 1 and 28 in male and female rats from 28-Day Study, with crystalline itraconazole, are summarized in Table 31.

TABLE 31
Plasma Cₘₐₓ and AUC₀₋ₗₐₛₜ for Itraconazole

| | | Cₘₐₓ (ng/mL) | | | | AUC_{0-Tlast} (ng · h/mL) | | | |
|---|---|---|---|---|---|---|---|---|---|
| | Dose level | Day 1 | | Day 28 | | Day 1 | | Day 28 | |
| Formulation | (mg/kg/day) | Males | Females | Males | Females | Males | Females | Males | Females |
| XXIII | 5.0 | 13.8 | 28.3 | 98.1 | 247 | 204 | 514 | 1020 | 5020 |
| XXIII | 14.5 | 39.6 | 134 | 110 | 550 | 473 | 2390 | 2140 | 11000 |

Despite differences in the absolute achieved doses between the two crystalline formulations, it is clear that the peak (Cₘₐₓ) and total (AUC₀₋ₗₐₛₜ) systemic exposure for PUR1920 is higher than that for any of the two crystalline formulations of Formulation XXIII, both after a single dose (Day 1) and repeat dosing (Day 28). Table 32 below summarizes the dose-normalized average Cₘₐₓ and AUC₀₋ₗₐₛₜ for itraconazole for each of the formulations from both 28-day studies using the target dose of 15 mg/kg/day from each study. Normalization was achieved by dividing the exposures measured by the actual achieved dose for each study on each day.

TABLE 32
Dose-normalized plasma Cₘₐₓ and AUC₀₋ₗₐₛₜ, for each of the crystalline
formulation.

| | Day 1 | | | | Day 28 | | | |
|---|---|---|---|---|---|---|---|---|
| | Cₘₐₓ | | AUC₀₋ₗₐₛₜ | | Cₘₐₓ | | AUC₀₋ₗₐₛₜ | |
| | (ng/mL/) | | (ng · h/mL) | | (ng/mL) | | (ng · h/mL) | |
| Formulation | M | F | M | F | M | F | M | F |
| XX | 17.40 | 28.18 | 135.50 | 509.09 | 18.61 | 68.95 | 117.22 | 1168.42 |
| XXIII | 1.96 | 5.08 | 28.15 | 134.27 | 7.68 | 43.47 | 138.06 | 658.68 |

The dose normalized Cₘₐₓ and AUC₀₋ₗₐₛₜ for itraconazole systemic exposure in rats on Day 1 were highest following exposure to Formulation XX. By Day 28, Formulation XX still generally showed higher dose normalized Cₘₐₓ and AUC₀₋ₗₐₛₜ relative to the crystalline formulation XXIII. These data demonstrate that, for a given achieved delivered lung dose. Formulation XX delivers itraconazole more quickly to the lungs and processes through the lungs faster than Formulation XXIII, exposing lung tissue and deep lung tissue to itraconazole more rapidly than Formulation XXIII.

### C. Results—Lung Pathology

In the first 28-Day study (Example 7), Formulation XX-related microscopic findings were present in respiratory tissues at ≥5 mg/kg/day. Minimal to slight granulomatous inflammation was present at all doses and macrophages and multinucleated giant cells frequently contained intracytoplasmic spicules. At the highest dose, where a 28-day recovery period was included, these only partially recovered. The pathology recorded was considered adverse at all doses due to its dispersed presentation and the fact that it did not fully resolve during the recovery period. The spicular formations noted in the pathology would appear to be itraconazole that, we theorize, are formed when the amorphous material supersaturates the lung lining fluid and interstitial space leading to crystallization of the API after multiple doses. Shorter duration exposure studies with the same formulation showed no such findings, leading us to believe short term dosing for acute treatment (e.g., one week, two weeks) could be advantageous.

In the second 28-Day Study (Example 9), Formulations XXI and XXIII were associated with minimal adverse accumulations of foamy macrophages in the lungs only at 40 mg/kg/day with Formulation XXI, the only formulation dosed at that level. Overall, the No Observed Adverse Effect Level (NOAEL) was approximately 15 mg/kg/day.

The systemic exposure, i.e., plasma levels of rats to itraconazole was highest following administration of Formulation XX. Systemic exposure was generally less following inhalation administration of Formulation XXIII, though by Day 28 of dosing the differences were less than after a single dose. The amorphous nature of the itraconazole in Formulation XX leads to increased solubility and rapid transit through the lung to the systemic circulation as evidenced by the significantly higher systemic exposure on Day 1.

### EXAMPLE 10

### In Vitro Dissolution Study of Dry Powder Formulations Containing Itraconazole

A. In vitro Dissolution Study

An in vitro model was utilized to provide a predictive test to understand the dissolution of itraconazole. Drug dissolution is a prerequisite for cellular uptake and/or absorption via the lungs. Hence, the dissolution kinetics of itraconazole plays a key role in determining the extent of its absorption from the respiratory tract. For dry particles containing itraconazole that are delivered to the respiratory tract as an aerosol, the fate of the itraconazole in those particles is dependent on their physicochemical properties. For the itraconazole in the aerosolized dry particles to exert a local effect in the lung, the dry particle must first undergo dissolution for the itraconazole to be present in the lung fluid and tissue to thereby act on a fungal infection. However, once dissolution of the itraconazole into the lung fluid has occurred, the itraconazole may further become available for permeation and systemic absorption. The rate of dissolution of itraconazole was predicted to be proportional to its solubility, concentration in surrounding liquid film and area of solid-liquid interface. Solubility is dependent on compound, formulation and physical form of the drug. The total liquid volume in the lung is 10-30 mL with a lining fluid volume corresponding to ca. 5 μL/cm², which may compromise the solubilization and subsequent absorption of poorly soluble molecules such as itraconazole.

The following in vitro dissolution model was used to understand the dissolution properties of itraconazole containing dry powder aerosols. The aerosol particles were collected at well-defined aerosol particle size distribution (APSD) cut-offs using the Next Generation Impactor (NGI) (Copley Scientific, UK), and then the dissolution behavior simulated using model lung fluid.

A UniDose™ (Nanopharm, Newport, United Kingdom) aerosol dose collection system combined with a modified next generation impactor (NGI) was used to uniformly deposit the impactor stage mass (ISM), which is defined as the dose collected on and below stage 2 of a next generation impactor, onto a suitable filter for subsequent dissolution studies in a USP V—Paddle over disk (POD) apparatus.

### B. Materials and Methods for the In Vitro Dissolution Study

The materials used in the study are shown in Table 21. The powder formulations, capsules and packaging materials were equilibrated at 22.5±2.5 and 30±5% RH. Formulations were encapsulated into size 3 HPMC capsules under the same conditions. The till weight for the powder preparations was 10 mg. The formulations were aerosolized from capsules in a unit-dose, capsule-based DPI device (RS01, Plastiape, Osnago, Italy).

One capsule of each formulation was aerosolized at 60 L/min (4 L inhaled volume) using the Plastiape RS01 dry powder inhaler (DPI). The aerosol dose was collected in the UniDose system. One milliliter of the suspension formulations was aerosolized into the cNGI at 15 L/min using a Micro Mist™ Nebuliser (Hudson RCI, Temecula, Calif., USA). The UniDose collection system was used to uniformly deposit the whole impactor stage mass (i.e., below stage 2 of an NGI) onto a glass microfiber filter membrane, which can be seen as where the circles (representing particles or droplets) deposit. The filter was placed into a disk cassette and dissolution studies were undertaken using 500 ml PBS pH 7.4±2.0% SDS in a USP Apparatus II POD (Paddle Over Disk, USP V) at 37° C. For all studies, sink conditions were maintained within the vessel. Samples were taken at specified time points and tested for drug content on an Agilent (Santa Clara, Calif., USA) 1260 Infinity series HPLC. Data has been presented as raw cumulative mass and cumulative mass percentage (%) at 240 minutes (mins).

TABLE 33
Formulations tested.

| Formulation | Description of Itraconazole |
|---|---|
| I | Dry powder formulation (amorphous itraconazole) |
| XV | Micro formulation with polysorbate 80 (Jet milling |
| | process #1) - Dry Powder Formulation |
| IX | Liquid Microsuspension Formulation |
| Pure ITZ | 100% API of Itraconazole as-received from |
| | manufacturer |

TABLE 34
Formulation

| | | | | Antifungal subparticle | |
|---|---|---|---|---|---|
| | | | | size (left column), and | |
| Formu- | Antifungal | Excipients | Stabilizer | range (right column) | |
| lation | (wt %) | (wt %) | (wt %) | (Dv50 nm) | |
| XV | Itraconazole | Sodium | PS80 | 1600 | 1200-1500 |
| | 50% | sulfate 35% | 5% | | |
| | | Leucine 10% | | | |

### C. Results of the UniDose POD Dissolution Studies of the Impactor Stage Mass (ISM) of the Formulations.

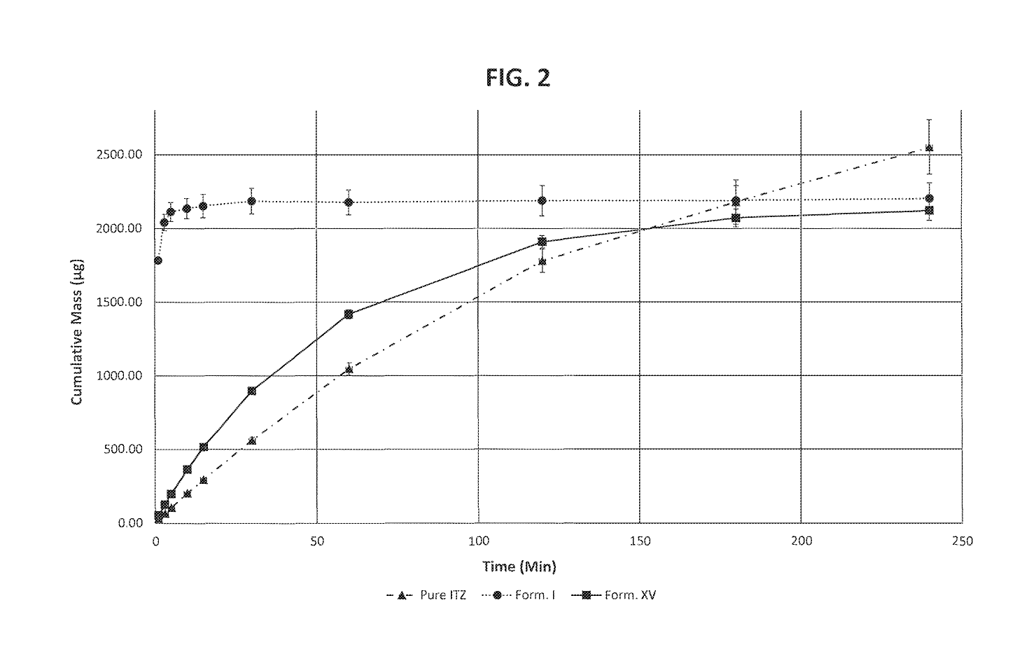

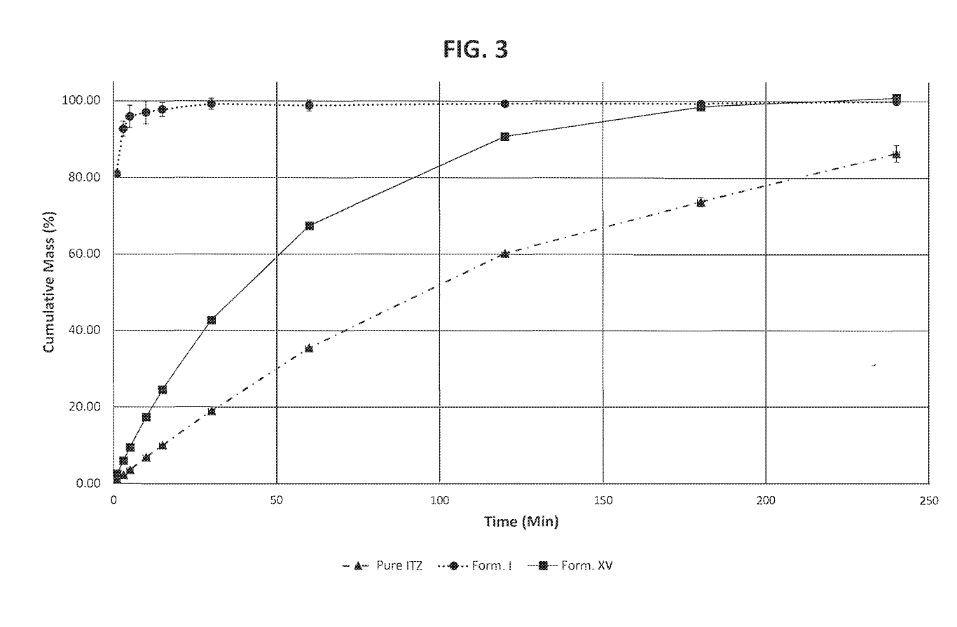

The raw cumulative mass and percentage cumulative mass dissolution plots of the ISM of formulations are shown in FIGS. 2 and 3, respectively. The UniDose ISM and dissolution half-life of each powder formulation is summarized in Table 21. Particle size of the itraconazole crystal in suspension and the specific surface area (SSA) of the itraconazole crystals estimated using the measured particle size distributions are also shown in Table 22.

Based on the cumulative mass data, the collected ISM of the formulations ranged between 2.1-2.6 mg itraconazole. These data suggested that the aerosolization efficiency of the formulations was approximately 50% based on the nominal dose, since the itraconazole loading in each particle was 50% and the nominal dose was 5 mg of itraconazole (10 mg of powder).The rate of dissolution of Formulation I, the amorphous itraconazole formulation, was the fastest and more than 80% of the drug had dissolved within the first time-point. Due to the rapid dissolution kinetics of formulation I it was not possible to calculate the dissolution half-life.

Based on the pharmacokinetic data shown in Example 5, Formulation I had the highest systemic exposure. This correlated with the dissolution data, which suggested that this formulation had rapid dissolution kinetics.

### EXAMPLE 11

### In Vitro Dissolution and Permeability Study of Dry Powder Formulations Containing Itraconazole

A. In vitro Dissolution and Permeability Study

A bio-relevant dissolution testing system was used based on mimicking the air-liquid interface at the respiratory epithelium interface using a cell-based in vitro method. A modified next generation impactor that incorporated cell culture plates onto collection stages (cNGI) was used to uniformly deposit materials onto the cell cultures. Dissolution and permeation of the drug through the epithelial cell monolayer was measured.

### B. Materials and Methods for the In Vitro Dissolution and Permeability Study

Epithelial cell monolayers grown at the air-liquid interface in Snapwell™ (Corning Costar, Massachusetts, USA) permeable insert were integrated into the cNGI. Calu-3 cell line (ATCC, LGC Standards, Teddington, UK) (passage 32-50) were grown in minimum essential medium (MEM) supplemented with non-essential amino acids, 10% (v/v) fetal bovine serum, 1% (v/v) penicillin-streptomycin and 1% (v/v) Fungizone antimycotic and maintained in a humidified atmosphere of 95%/5% Air/CO₂, respectively, at 37° C. Cells were seeded on to Snapwell inserts at a density of 5×10⁵ cells·cm⁻² and cultured under air-interfaced conditions from day 2 in culture for 12 days. The transepithelial electrical resistance (TEER) was measured using an EVOM2 chopstick electrode connected to an EVOM2 Epithelial Voltohmmeter (World Precision Instruments, Hitchin, United Kingdom) and monolayers with a TEER above 450 Ω·cm² were deemed confluent.

Snapwells containing Calu-3 ALI cells were transferred to a modified NGI cup and placed into stage 4 of the NGI (Copley Scientific, Nottingham, UK). A single capsule of the powder formulations was aerosolized into the cNGI at 60 L/min for 4 seconds. One milliliter of the suspension formulations was aerosolized into the cNGI at 15 L/min using a Micro Mist Nebulizer (Hudson RCI, Temecula, Calif., USA).

The materials used in the study are shown in Table 21. The powder formulations, capsules and packaging materials were equilibrated at 22.5±2.5° C. and 30±5% RH. Formulations were encapsulated into size 3 HPMC capsules under the same conditions. The fill weight for the powder preparations was 10 mg. The formulations were aerosolized from capsules in a unit-dose, capsule-based DPI device (RS01, Plastiape, Osnago, Italy). One capsule of each formulation was aerosolized at 60 L/min (4 L inhaled volume) using the Plastiape RS01 dry powder inhaler (DPI).

Post-dosing of the dose on to the Snapwells from stage 4, the Snapwells were transferred to 6-well plates, which contained 2mL of PBS pH 7.4±2.0% SDS maintained at 37° C. Basolateral samples were taken at different time points and drug content was measured on an Agilent (Santa Clara, Calif., USA) 1260 Infinity series HPLC. Total dose delivered to the cells was measured from the total amount of drug dissolved over the time-course and from lysing cells post experimentation.

C. Results of the cNGI Integrated Dissolution and Permeability Studies of Powder Formulations of Itraconazole

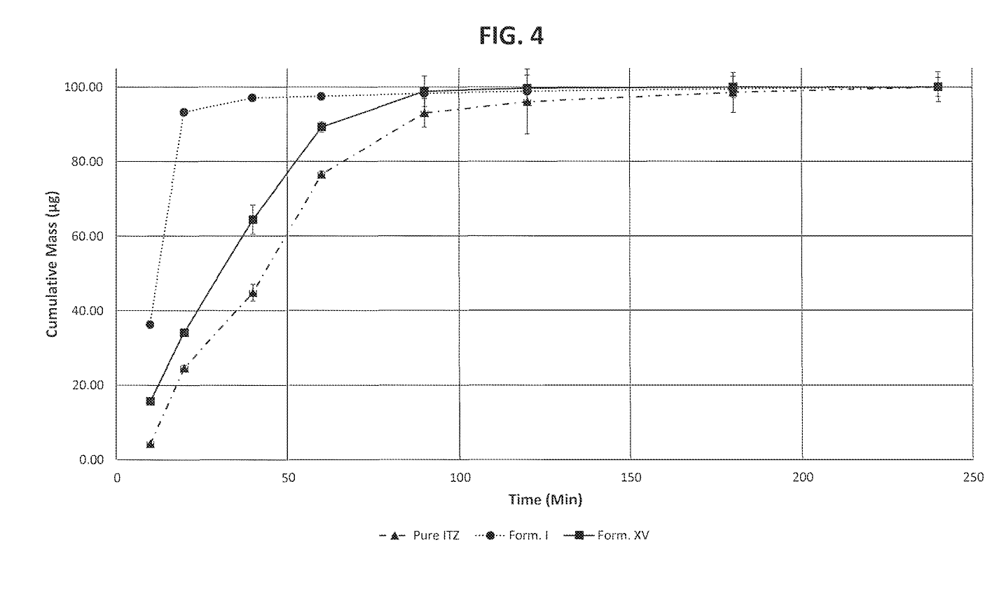

The cumulative mass percent (%) of the total recovered dose plots of the powder formulations of itraconazole delivered to the cells on stage 4 are shown in FIG. 4. These data suggested differences between the dissolution and permeability kinetics of the different formulations. The as-received Pure ITZ had slower dissolution and permeability kinetics than the other formulations, whilst Formulation I had the fastest dissolution and permeability kinetics.

To understand the cNGI data for the different formulations, we utilised the data to calculate the rate of diffusion of the drug substance by taking into consideration loaded dose differences. This was done using the following equation:

$$Rate of Diffusion = \frac{J}{A {C}_{0}}$$

where J is the flux (gradient of the cNGI dissolution/permeability profile), A is the area of the barrier and Co is the loaded dose. These data are summarised in Table 35, which shows that the rate of diffusion for the formulations followed the rank order:

I>XV>Pure ITZ

TABLE 35
Particle size and rate of diffusion
of each powder formulation below.

| | PSD of Itraconazole | Rate of Diffusion |
|---|---|---|
| Formulations | Crystal (nm) | (cm/s) |
| Pure ITZ | Not Known | 1.81 |
| XV | 1600 | 2.52 |
| I | Not Known | 7.01 |

Based on the pharmacokinetic data shown in Example 5, Formulation I (the amorphous itraconazole formulation) had the highest systemic exposure. This correlated with the rate of diffusion of this formulation, which suggested that this formulation had rapid dissolution and permeation kinetics.

### Summary In-Vitro and In-Vivo Example Summary

The investigation of the effects of the physical form of itraconazole within the dry powder formulations involved an iterative progression through in-vitro dissoluation and permeability studies and in-vivo single and multiple dose pharmacokinetic and toxicity studies. The in-vitro dissolution studies demonstrated that the physical form of itraconazole plays an important role in determining the rate of dissolution as well as the rate at which the delivered material would be expected to pass through the lung and into the systemic circulation. These data demonstrate the ability to control key aspects of both the lung and systemic exposure to allow the modulation of both efficacy as well as potentially the modulation of adverse findings. The example summarizing the 28-day inhalation toxicity studies further demonstrated that the exposure kinetics with amorphous itraconazole in the dry powder formulations, in terms of systemic exposure, are retained over multiple days of dosing.

Future studies will likely focus on ways to avoid toxicity issues seen with the amorphous formulations, including smaller doses or short-term dosing (e.g., one week, two week) instead of more long-term dosing.

While this invention has been particularly shown and described with references to example embodiments thereof, it will be understood by those skilled in the art that various changes in form and details may be made therein without departing from the scope of the invention encompassed by the appended claims.

1. A dry powder comprising respirable dry particles that comprise amorphous itraconazole in an amount of about 45% to about 75%, sodium sulfate in an amount of about 10% to about 55%, and optionally one or more other excipients, wherein all percentages are weight percentages on a dry basis and all the components of the respirable dry particles amount to 100%.

2. The dry powder of claim 1, wherein the one or more other excipients are selected from the group consisting of leucine, mannitol, or combinations thereof.

3. The dry powder of claim 1, wherein the amorphous itraconazole is about 45% to about 55%.

4. (canceled)

5. The dry powder of claim 1, wherein the sodium sulfate is in an amount of about 30% to about 40%.

6. (canceled)

7. The dry powder of claim 1, wherein the one or more other excipients total about 5% to about 25%.

8. The dry powder of claim 1, wherein the amorphous itraconazole is about 50%, the sodium sulfate is in an amount of about 35%, the one or more other excipients total about 15%.

9. The dry powder of claim 1, wherein the one or more other excipients is leucine.

10. (canceled)

11. A dry powder comprising respirable dry particles that comprise amorphous itraconazole in an amount of about 45% to about 55%, sodium chloride in an amount of about 30% to about 40%, and leucine in an amount of about 10% to about 20%, wherein all percentages are weight percentages on a dry basis and all the components of the respirable dry particles amount to 100%.

12. A dry powder comprising respirable dry particles that comprise amorphous itraconazole in an amount of about 50%, sodium chloride in an amount of about 35%, and leucine in an amount of about 15%, wherein all percentages are weight percentages on a dry basis and all the components of the respirable dry particles amount to 100%.

13. (canceled)

14. (canceled)

15. The dry powder of claim 1, wherein the respirable dry particles have a volume median geometric diameter (VMGD) about 10 microns or less.

16. The dry powder of claim 1, wherein the respirable dry particles have a volume median geometric diameter (VMGD) about 5 microns or less.

17. The dry powder of claim 1, wherein the respirable dry particles have a tap density of about 0.2 g/cc or greater.

18. (canceled)

19. The dry powder of claim 1, wherein the dry powder has an MMAD of between about 1 micron and about 5 microns and a FPF of the total dose less than 3.4 microns of about 25% or more.

20. The dry powder of claim 1, wherein the dry particles have a 1/4 bar dispersibility ratio (1/4 bar) of less than about 1.5 as measured by laser diffraction.

21-24. (canceled)

25. The dry powder of claim 1, wherein the dry powder is delivered to a patient with a capsule-based passive dry powder inhaler.

26. The dry powder of claim 1, wherein the respirable dry particles have a capsule emitted powder mass of at least 80% when emitted from a passive dry powder inhaler that has a resistance of about 0.036 sqrt(kPa)/liters per minute under the following conditions; an inhalation flow rate of 30 LPM for a period of 3 seconds using a size 3 capsule that contains a total mass of 10 mg, said total mass consisting of the respirable dry particles, and wherein the volume median geometric diameter of the respirable dry particles emitted from the inhaler as measured by laser diffraction is 5 microns or less.

27. A method for treating a fungal infection comprising administering to the respiratory tract of a patient in need thereof an effective amount of a dry powder of claim 1.

28. A method for treating a fungal infection in a patient with asthma, cystic fibrosis or an immunocompromised patient comprising administering to the respiratory tract of the asthma or cystic fibrosis patient an effective amount of a dry powder of claim 1.

29. A method for treating aspergillosis or allergic bronchopulmonary aspergillosis (ABPA) comprising administering to the respiratory tract of a patient in need thereof an effective amount of a dry powder of claim 1.

30. (canceled)

31. A method for treating or reducing the incidence or severity of an exacerbation of a respiratory disease comprising administering to the respiratory tract of a patient in need thereof an effective amount of a dry powder of claim 1, wherein the exacerbation is a fungal infection.

32. (canceled)
